(19) United States
(12) Patent Application Publication
(10) Pub. No.: US 2021/0109111 A1
(43) Pub. Date: Apr. 15, 2021

(54) NEW BIOMARKERS OF HUMAN SKIN AGING

(71) Applicant: Université Grenoble Alpes, Saint Martin d'Hères (FR)

(72) Inventors: Sandrine BOURGOIN-VOILLARD, GRENOBLE (FR); Sylvia Marie-Louise LEHMANN, GRENOBLE (FR); Walid RACHIDI, MEYLAN (FR); Michel SEVE, MEYLAN (FR)

(21) Appl. No.: 16/464,517

(22) PCT Filed: Nov. 24, 2017
(86) PCT No.: PCT/EP2017/080414

§ 371 Date: May 28, 2019

(30) Foreign Application Priority Data
Nov. 25, 2016 (EP) 16306561.8

(51) Int. Cl. G01N 33/68; G01N 33/50

(52) U.S. Cl. CPC G01N 33/6881; G01N 33/5044; G01N 33/5023

(57) ABSTRACT

The present invention refers to an in vitro method to determine if the skin of a subject presents signs of physiological aging, a method of cosmetic and a method to identify a substance that is capable of reducing or reversing the visible signs of physiological skin aging. The invention further refers to a kit comprising capture ligands and a use of said kit for determining in a skin sample the expression level of the markers of skin aging that are identified in context of the present invention.

The present invention refers to an in vitro method to determine if the skin of a subject presents signs of physiological aging, a method of cosmetic treatment and a method to identify a substance that is capable of reducing or reversing the visible signs of physiological skin aging. The invention further refers to a kit comprising capture ligands and a use of said kit for determining in a skin sample the expression level of the markers of skin aging that are identified in context of the present invention.

Life expectancy in developed countries over the past two centuries has considerably increased and if this trend continues through the 21st century, most babies born since 2000 in such countries will reach 100 years. Also it is expected that by 2030, one in eight people worldwide will be 65 or above and the global aging of the population will lead to several societal, economical and medical challenges (Christensen et al., 2009, Lancet 374, 1196-1208).

Aging is a complex process influenced by multiple genetic and environmental factors and is characterized by a progressive decline in multiple physiological functions. Skin, like other organs, is affected by aging that can be accelerated by environmental factors such as UV radiation. Intrinsic skin aging, also called chronological aging, is observed in sun nonexposed skin and reflects the aging process of the entire organism (Makrantonaki et al., 2007, Exp. Gerontol. 42, 879-886). Thereby, skin is an interesting alternative approach to decipher the intrinsic aging process as it is easily accessible compared to internal organs or tissues. Skin undergoes several morphological and physiological changes with intrinsic aging such as fine wrinkles formation, thinning of the epidermis and dermis, increased vulnerability and fragility, dryness, loss of elasticity, disturbed barrier function . . . (Zouboulis and Makrantonaki, 2011, Clin. Dermatol. 29, 3-14). The underlying mechanisms of intrinsic aging are multiple: cellular senescence and decreased proliferative capacity; shortening of the telomeres; increase in DNA damage and reduction in DNA repair processes; mitochondrial and genomic DNA mutations; hormonal decline and oxidative stress (Poljšak et al., 2012, Acta Dermatovenerol. Alp. Pannonica Adriat. 21, 33-36) (Makrantonaki and Zouboulis, 2007, Dermatol. Basel Switz. 214, 352-360).

Skin aging is a complex process and a lot of efforts have been made to better understand the biology of skin aging and identify new and specific targets that could help to diagnose, prevent and treat skin aging and related pathologies. Over the last decade, several transcriptomic studies have investigated the effect of aging on gene expression in several organism models and in humans (Zahn et al., 2007, PLoS Genet. 3, e201) (Zahn and Kim, 2007, Curr. Opin. Biotechnol. 18, 355-359).

Concerning skin aging, only four studies have been conducted in humans. The first study shows that genes differently expressed in elderly and young human male skin, were involved in various cellular processes such as metabolism, signal transduction, apoptosis, regulation of transcription (Lener et al., 2006, Exp. Gerontol. 41, 387-397). More recently, a study has compared the gene expression profile from sun nonexposed skin in both gender depending on aging. There was a significant different response in both genders with aging, with only 39 genes commonly dysregulated with 4 of them regulated in opposite manner in both genders. From these results, the WNT signaling pathway has emerged as the major downregulated pathway with aging in both sexes (Makrantonaki et al., 2012, PLoS ONE 7). And lately 75 differentially expressed genes according to age status in human epidermis were identified (Raddatz et al., 2013, Epigenetics Chromatin 6, 36). Pathway analysis revealed that these genes were mainly involved in cell migration, cancer, dermatological diseases and cell proliferation. Also genes involved in the development of the epidermis were significantly enriched and an overall downregulation of keratinocytes differentiation was observed.

Proteins are the workhorses of the cell and the main effectors of numerous cellular processes. Quantitative mass spectrometry based proteomics has proven its utility for the description of protein dynamics in order to decipher complex processes and describe normal states and pathological states of cells. Those recent technological advances, in particular in mass spectrometry, have allowed for large-scale surveys of the proteome. These studies changed the general understanding of protein-expression regulation and demonstrated that the concentration of gene transcripts such as mRNA concentrations do not necessarily reflect the concentrations and activities of the corresponding proteins.

Relatively few studies have used proteomics to investigate skin aging and they all used a two-dimensional gel electrophoresis approach which leads to a lower coverage of the proteome. As a result only a few dysregulated proteins were identified whereas gel-free approaches could provide a proteomic signature of aging (Laimer et al., 2010, Exp. Dermatol. 19, 912-918) (Delattre et al., 2012, Exp. Dermatol. 21, 205-210) (Gromov et al., 2003).

Skin is composed of several cell layers and previous studies have investigated the protein expression level in whole skin or in stratum corneum samples. But the different cell layers can behave very differently and exhibit a different response to aging. Keratinocytes are the major components of the epidermis which is the most superficial and accessible layer of the skin. Focusing on keratinocytes in culture helps to gain a deeper insight in skin aging and to have access to the proliferative compartment of the epidermis.

The inventors of the present invention investigated the changes in the protein expression profile in human primary keratinocytes derived from sun nonexposed skin obtained from young and elderly caucasian women. Considering hormones as one major factor affecting aging, the age categories were chosen so that circulating hormones are at their higher level for young and at their lowest for elderly women.

The inventors of the present invention identified 58 proteins which expression was significantly dysregulated that are putative candidate biomarkers for intrinsic skin aging using a quantitative proteomic approach of young and elderly primary human keratinocytes.

In particular, the inventors of the present invention identified the proteins TUBB3, HMGA2 and HMGN1 as particularly well suited biomarkers in order to identify skin that presents signs of physiological aging.

### SUMMARY OF THE INVENTION

The inventors of the present invention identified fifty eight proteins that are significantly differentially expressed in older skin versus younger skin (pValue <0.05).

From those 58 biomarkers, 40 were downregulated and 18 were upregulated with aging as identified by quantitative proteomics. The diagnostic value of two of these markers TUBB3 and HMGA2 was further confirmed by Western Blot analysis using skin samples from other subjects.

The present invention thus concerns an in vitro method to determine if the skin of a subject presents signs of physiological skin aging comprising the steps of

a) determining in a skin sample of said subject the expression level of a first protein encoded by a gene selected from the group of genes consisting of TUBB3, HMGA2 and HMGN1, preferably TUBB3 and HMGA2, more preferably TUBB3, and the expression level of at least one further protein encoded by a gene selected from the group of genes consisting of HMGN1, HIST1H2BK, PPFIA2, COXSA, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7, TUBB3, HMGA2, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13,

b) determining if the skin presents signs of physiological skin aging.

The present invention further refers to a method of cosmetic treatment capable of reducing or reversing the visible signs of physiological skin aging in a subject comprising the steps of

a) determining in a skin sample of said subject the expression level of a first protein encoded by a gene selected from the group of genes consisting of TUBB3, HMGA2 and HMGN1, preferably TUBB3 and HMGA2, more preferably TUBB3, and the expression level of at least one further protein encoded by a gene selected from the group of genes consisting of HMGN1, HIST1H2BK, PPFIA2, COX5A, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7, TUBB3, HMGA2, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13,

b) deducing from the expression level of said first protein and the expression level of said at least one further protein determined in step a) if the skin presents signs of physiological skin aging, and

c) if the skin is determined as presenting signs of physiological aging, treating said subject with a cosmetic composition that reduces or reverses the visible signs of physiological skin aging.

The invention further refers to a method to identify a substance that is capable of reducing or reversing the visible signs of physiological skin aging comprising the steps of

a) treating a skin sample with a candidate substance,

b) determining in the skin sample of step a) the expression level of a first protein encoded by a gene selected from the group of genes consisting of TUBB3, HMGA2 and HMGN1, preferably TUBB3 and HMGA2, more preferably TUBB3, and the expression level of at least one further protein encoded by a gene selected from the group of genes consisting of HMGN1, HIST1H2BK, PPFIA2, COX5A, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7, TUBB3, HMGA2, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13,

c) comparing the expression level of said first protein and said at least one further protein with the expression level of said first protein and said at least one further protein in a skin sample that has not been treated with said candidate substance,

d) identifying the candidate substance as a substance that reduces or reverses the visible signs of physiological skin aging.

The invention further refers to a kit comprising
at least one capture ligand for determining the expression level of a first protein encoded by a gene selected from the group of genes consisting of TUBB3, HMGA2 and HMGN1, preferably TUBB3 and HMGA2, more preferably TUBB3, and
at least one capture ligand for determining the expression level of at least one further protein encoded by a gene selected from the group of genes consisting of HMGN1, HIST1H2BK, PPFIA2, COXSA, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7, TUBB3, HMGA2*, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13.

The invention further refers to the use of a kit as defined herein above for determining in a skin sample the expression level of one first protein encoded by a gene selected from the group of genes consisting of TUBB3, HMGA2 and HMGN1, preferably TUBB3 and HMGA2, more preferably TUBB3, and the expression level of at least one further protein encoded by a gene selected from the group of genes consisting of HMGN1, HIST1H2BK, PPFIA2, COXSA, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7, TUBB3, HMGA2, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13.

### DETAILED DESCRIPTION OF THE INVENTION

“Aging” in context of the present invention refers to the cutaneous effects of aging and is herein referred to as “skin aging”.

“Skin aging” also referred to as “physiological skin aging” can be described clinically as skin having features such as, wrinkles, sunspots, uneven skin color, and sagging skin. In addition to inherited genetic traits, a multitude of other factors can modify the aging process, such as hormonal status and climatic, working, social, and cultural conditions. It will be understood by the skilled in the art, that the effects of aging on the skin are influenced by both intrinsic and extrinsic factors. Similar to other organs, the human skin undergoes progressive functional decline due to the accumulation of molecular damage. Oxidative stress and molecular damage contribute to both intrinsic aging, also called chronological aging and aging as a consequence of environmental factors, also called extrinsic aging. As a consequence, aged skin or “older skin” exhibits many differences in comparison to youthful skin and also has a marked susceptibility to dermatologic disorders due to the structural and physiologic changes that occur with time.

It will be further understood by the skilled in the art that beside chronological aging and extrinsic aging, the occurrence of a skin disease may induce physiological aging of the skin and may be referred to as “skin disease derived skin aging”.

Accordingly, “skin aging” or “physiological skin aging” as used in context of the present invention thus refers to chronological aging, extrinsic aging and/or aging due to a skin disease, preferably chronological aging and/or extrinsic aging.

In one particular embodiment, skin aging is skin disease derived skin aging.

“Chronological aging” or “intrinsic aging” reflects the genetic background of an individual and occurs with the passage of time. Intrinsically aged skin is typically smooth and unblemished. With chronological aging alone, elderly will exhibit thin skin with fine wrinkles, fat atrophy with soft tissue redistribution, and bone remodeling. It is known to the skilled in the art, that people of color exhibit less severe intrinsic facial aging with signs appearing a decade later than lighter skin types.

“Extrinsic aging” relates to environmental exposures, health, and lifestyle that are associated with individual habits, such as sun exposure, tobacco use, diet, and exercise and thus includes for example photoaging. Cumulative sun exposure is the most important extrinsic factor in skin aging. In some skin types dyspigmentation is one of the most common features of photoaging. Common clinical signs of photoaging include lentigines, rhytides, telangiectasias, dark spots, and loss of elasticity. It is known to the skilled in the art, that skin of color is less susceptible to sun-induced damage so these clinical manifestations of aging are less severe and typically occur 10 to 20 years later than those of age-matched white counterparts. Other extrinsic factors, such as smoking, excessive alcohol, and poor nutrition, can also contribute to skin aging.

Non-limiting examples of a “skin diseases” that might induce physiological aging of the skin are for example atopic dermatitis, seborrheic keratitis, epidermolysis bullosa acquisita, psoriasis, skin alterations in lupus erythematosus, dermatomyositis, scleroderma, chronic acne, chronic cellulites, pruritus and abnormal or defective scar formation in diabetes and premature aging diseases.

“Premature aging diseases” herein refers to diseases that are characterized by premature skin aging and includes but is not limited to Hutchinson-Gilford Progeria syndrom (HGPS), Atypical progeria syndromes (APS), Mondibuloacral dysplasia (MAD), Werner syndrome (WS), Bloom syndrome (BS), Rothmund-Thomson syndrome (RTS), Cockayne syndrome (CS), Xeroderma Pigmentosum (XP), Trichothiodystrophy (TTD), Fanconi Anemia (FA), Ataxia telangiectasia (AT) and dyskeratosis congenita (DC).

In accordance with the above “signs of skin aging” include, but are not limited to, all outward visibly and tactilely perceptible manifestations as well as any other macro or micro effects due to skin aging. These signs may result from processes which include, but are not limited to, the development of textural discontinuities such as wrinkles and coarse deep wrinkles, fine lines, skin lines, crevices, bumps, large pores (e.g., associated with adnexal structures such as sweat gland ducts, sebaceous glands, or hair follicles), or unevenness or roughness, loss of skin elasticity (loss and/or inactivation of functional skin elastin), sagging (including puffiness in the eye area and jowls), loss of skin firmness, loss of skin tightness, loss of skin recoil from deformation, discoloration (including undereye circles), blotching, sallowness, hyperpigmented skin regions such as age spots and freckles, keratoses, abnormal differentiation, hyperkeratinization, elastosis, collagen breakdown, and other histological changes in the stratum corneum, dermis, epidermis, the skin vascular system (e.g., telangiectasia or spider vessels), and underlying tissues (e.g., fat and/or muscle), especially those proximate to the skin;

The “skin” is the largest human organ comprising about one sixth of total body weight. The skin performs a complex role in human physiology: serves as a barrier to the environment, and the sebum produced by some of its glands (sebaceous) have anti-infective properties. The skin acts as a channel for communication to the outside world, protects us from water loss, friction wounds, and impact wounds and uses specialized pigment cells to protect us from ultraviolet rays of the sun. Skin produces vitamin D in the epidermal layer, when it is exposed to the sun's rays. The skin helps regulate body temperature through sweat glands and helps regulate metabolism. The skin consists of three functional layers: Epidermis, the Dermis (or corium) and the Subcutis (or hypodermis). Accordingly, in one embodiment, skin refers to the epidermis, the dermis and the subcutis, preferably to the epidermis and the dermis, more preferably to the epidermis.

Accordingly, the “skin sample” as referred to in context of the present invention preferably comprises the epidermis, the dermis and the subcutis, preferably the epidermis and the dermis, more preferably the epidermis.

“Epidermis” as used herein refers to the outer layer of skin, and is divided into five strata, which include the: stratum corneum, stratum lucidum, stratum granulosum, stratum spinosum, and stratum basale. The stratum corneum contains many layers of dead, anucleated keratinocytes that are essentially filled with keratin. The outermost layers of the stratum corneum are constantly shed, even in healthy skin. The stratum lucidum contains two to three layers of anucleated cells. The stratum granulosum contains two to four layers of cells that are held together by desmosomes that contain keratohyaline granules. The stratum spinosum contains eight to ten layers of modestly active dividing cells that are also held together by desmosomes. The stratum basale contains a single layer of columnar cells that actively divide by mitosis and provide the cells that are destined to migrate through the upper epidermal layers to the stratum corneum. Thus, the predominant cell type of the epidermis is the keratinocyte. These cells are formed in the basal layer and exist through the epidermal strata to the granular layer at which they transform into the cells known as corneocytes or squames that form the stratum corneum. During this transformation process, the nucleus is digested, the cytoplasm disappears, the lipids are released into the intercellular space, keratin intermediate filaments aggregate to form microfibrils, and the cell membrane is replaced by a cell envelope made of cross-linked protein with lipids covalently attached to its surface.

In some embodiments, the methods of the invention further comprise a step 0) of obtaining a skin sample.

In one embodiment, the skin sample in context of the present invention comprises skin cells. The skin sample in context of the present invention may therefore also be referred to as skin sample cells.

In one embodiment, the skin sample cells are keratinocytes, preferably primary keratinocytes.

Accordingly, in one embodiment, the skin sample in context of the present invention comprises keratinocytes, preferably primary keratinocytes.

Methods to obtain a skin sample are generally known to the skilled in the art. In one example the skin sample is typically obtained using reconstructed skin, in particular 3D reconstructed skin, or skin biopsies followed by isolation and culture of the skin cells comprised in said skin sample, for example, followed by isolation and culture of primary keratinocytes.

In one example, skin biopsies of typically sun protected nonexposed skin were obtained after plastic mammary surgery and human primary keratinocytes were cultured in KSFM medium supplemented with 25 μg/mL BPE and 0.9 ng/mL EGF.

In one embodiment, the skin sample derived from protected nonexposed skin.

In one embodiment, the methods of the invention comprise a further step of preparing the skin sample prior to determining the protein expression level. Said preparation usually refers in context of the present invention to a protein extraction.

Accordingly, in one embodiment, the proteins are extracted from the skin sample, preferably the skin sample cells, prior to determining protein expression levels in context of the methods of the present invention.

Accordingly, in one embodiment the skin sample refers to a protein extract of said skin sample, preferably, a protein extract of the skin sample cells.

In one example, typically frozen pellets of skin cells of a skin sample are typically lysed for 30 minutes at, for instance, 4° in a solution containing, for example, 40 mM HEPES PH 7.4, 100 mM NaCl, 1 mM EDTA, 0.02% Triton, 0.02% Sodium Deoxycholate, 0.2 mM TCEP, and protease and phosphatase inhibitor cocktail (PhosSTOP) from Roche. Typically, lysis is achieved by short sonication on ice and the lysates are cleared, for example, by centrifugation at 14,000 rpm for 20 minutes at 4°.

“Keratinocytes” are the predominant cell type in the epidermis, the outermost layer of the skin, constituting 90% of the cells found there. Keratinocytes may be isolated by methods known to the skilled in the art for further analysis.

Accordingly, in one example the skin sample comprises at least 50% keratinocytes, such as 60%, 65%, 70%, 75%, 80%, 85% keratinocytes.

“Primary keratinocytes” herein refers to isolated keratinocytes from human skin sample.

The wording “biomarker” as generally used refers to any biological molecules (genes, proteins, lipids, metabolites) that, singularly or collectively, reflect the current or predict future state of a biological system. Proteins, as biomarkers, have the advantage that they have a longer half-life time than, typically, m-RNA, and their concentration is thus more stable over a given time period. As used herein, different proteins as listed herein below in the section “methods of the invention” are identified as biomarkers that are indicators for physiological skin aging. Non-limiting examples of biomarkers as identified in context of the present invention are the proteins encoded by the genes TUBB3, HMGA2 and/or HMGN1. The biomarkers of the invention are further described herein below in context of the methods of the invention.

“Gene” used herein may be a genomic gene comprising transcriptional and/or translational regulatory sequences and/or a coding region and/or non-translated sequences (e.g., introns, 5′- and 3′-untranslated sequences). The coding region of a gene may be a nucleotide sequence coding for an amino acid sequence or a functional RNA, such as long and short non-coding RNA (as an example: tRNA, rRNA, catalytic RNA, and miRNA). A gene may also be an mRNA or cDNA corresponding to the coding regions (e.g., exons and miRNA) optionally comprising 5′- or 3′-untranslated sequences linked thereto. A gene may also be an amplified or synthetic nucleic acid molecule comprising all or a part of the coding region and/or 5′- or 3′-untranslated sequences linked thereto.

### Subject

In the context of the invention, “subject” refers to an animal, preferably a non-human or human mammal. Examples of non-human mammals include rodents and primates. Most preferably, the subject is a human.

A “human” may be further distinct into different races. The 3 major human races are the Caucasian race including Aryans, Hamites, Semites, the Mongolian race including for example northern Mongolian, Chinese and Indo-Chinese, Japanese and Korean, Tibetan, Malayan, Polynesian, Maori, Micronesian, Eskimo, American Indian, the Negroid race including, for example, African, Hottentots, Melanesians/Papua, “Negrito”, Australian Aborigine, Dravidians, Sinhalese.

As known by the skilled in the art the cutaneous effects of aging as defined herein above are influenced by both intrinsic and extrinsic factors and often are varied based on ethnic origin given underlying structural and functional differences.

Accordingly in one embodiment the human is Caucasian, Mongolian or Negro, preferably Caucasian.

Accordingly, in one preferred embodiment the subject is Caucasian.

The term “Caucasian” is commonly used to refer to the combination of physical attributes of individuals of European, Northern African, and Southwest Asian ancestry.

This group of “Caucasian subjects” comprises those of lightly pigmented skin, demonstrated by small, aggregated melanosomes along with reduced amounts of melanin. The decreased epidermal melanin component predisposes Caucasians to develop earlier signs of photoaging than other populations. European Americans with low constitutive pigmentation have considerably higher burn response and lower tanning ability compared with Hispanics and East Asians. In addition, Caucasian skin is exemplified by a thinner and less cohesive stratum corneum, reduced skin extensibility, along with loss of collagen and disorganization of the elastic fibers in the dermis with increasing age. These attributes result in clinically fragile skin and contribute to the aging process.

Hormones are one factor of aging and the amount and type of hormones differ between male and female objects of the same range of age.

Accordingly in one embodiment the human herein refers to men or women, preferably women.

Accordingly, in one embodiment, the subject is a woman.

In one embodiment, the age of the subject is from 18 to 95, preferably 32 to 80, such as 45 to 80, more preferably 45 to 80.

### Methods of the Invention

The “at least one further protein” in context of the inventions refers to one further protein, two further proteins, three further proteins, four further proteins, five further proteins, six further proteins, seven further proteins, eight further proteins, nine further proteins or ten further proteins, preferably to one further protein, two further proteins, three further proteins, four further proteins.

The at least one further protein in context of the present invention is encoded by a gene selected from the group of genes consisting of HMGN1, HIST1H2BK, PPFIA2, COXSA, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7, TUBB3, HMGA2, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13.

The amino acid sequences of these 58 biomarkers used in context of the invention are available from the UniProtKB database under the Accession code as listed herein below and as accessible on 15 Jan. 2016.

| Accession/ | | | |
|---|---|---|---|
| variants | ID | Description | Gene |
| O60814 | H2B1K_HUMAN | Histone H2B type 1-K | HIST1H2BK |
| O75334- | LIPA2_HUMAN | Liprin-alpha-2 | PPFIA2 |
| [2-6] | | | |
| P20674 | COX5A_HUMAN | Cytochrome c oxidase sub- | COX5A |
| | | unit 5A, mitochondrial | |
| P04732 | MT1E_HUMAN | Metallothionein-1E | MT1E |
| P05204 | HMGN2_HUMAN | Non-histone chromosomal | HMGN2 |
| | | protein HMG-17 | |
| Q15075 | EEA1_HUMAN | Early endosome antigen 1 | EEA1 |
| Q9UKY7- | CDV3_HUMAN | Protein CDV3 homolog | CDV3 |
| [2] | | | |
| O75152 | ZC11A_HUMAN | Zinc finger CCCH domain- | ZC3H11A |
| | | containing protein 11A | |
| P17096 | HMGA1_HUMAN | High mobility group | HMGA1 |
| | | protein HMG-I/HMG-Y | |
| P06454- | PTMA_HUMAN | Prothymosin alpha | PTMA |
| [2] | | [Cleaved into: | |
| | | Prothymosin alpha, N- | |
| | | terminally processed; | |
| | | Thymosin alpha-1] | |
| Q8NC51- | PAIRB_HUMAN | Plasminogen activator | SERBP1 |
| [3] | | inhibitor 1 RNA-binding | |
| | | protein | |
| Q13442 | HAP28_HUMAN | 28 kDa heat-and acid- | PDAP1 |
| | | stable phosphoprotein | |
| P63313 | TYB10_HUMAN | Thymosin beta-10 | TMSB10 |
| O00233 | PSMD9_HUMAN | 26S proteasome non- | PSMD9 |
| | | ATPase regulatory | |
| | | subunit 9 | |
| P05114 | HMGN1_HUMAN | Non-histone chromosomal | HMGN1 |
| | | protein HMG-14 | |
| P62158 | CALM_HUMAN | Calmodulin | CALM1 |
| P02795, | MT1G_HUMAN, | Metallothionein-1G, | MT1G, |
| P13640- | MT1X_HUMAN, | Metallothionein-1X, | MT1X, |
| [2], | MT2_HUMAN | Metallothionein-2 | MT2A |
| P80297 | | | |
| P67936 | TPM4_HUMAN | Tropomyosin alpha-4 chain | TPM4 |
| P22528 | SPR1B_HUMAN | Cornifin-B | SPRR1B |
| O92538- | GBF1_HUMAN | Golgi-specific brefeldin | GBF1 |
| [2, 3] | | A-resistance guanine | |
| | | nucleotide | |
| | | exchange factor 1 | |
| P51858 | HDGF_HUMAN | Hepatoma-derived growth | HDGF |
| | | factor | |
| P61604 | CH10_HUMAN | 10 kDa heat shock | HSPE1 |
| | | protein, mitochondrial | |
| P07108- | ACBP_HUMAN | Acyl-CoA-binding protein | DBI |
| [2-5] | | | |
| Q9C030- | TRIM6_HUMAN | Tripartite motif-containing | TRIM6 |
| [2] | | protein 6 | |
| P20962 | PTMS_HUMAN | Parathymosin | PTMS |
| Q9GZP8 | IMUP_HUMAN | Immortalization up- | IMUP |
| | | regulated protein | |
| Q9H299 | SH3L3_HUMAN | SH3 domain-binding | SH3BGRL3 |
| | | glutamic acid-rich- | |
| | | like protein 3 | |
| P62857 | RS28_HUMAN | 40S ribosomal protein S28 | RPS28 |
| P16949- | STMN1_HUMAN | Stathmin | STMN1 |
| [2] | | | |
| P02765 | FETUA_HUMAN | Alpha-2-HS-glycoprotein | AHSG |
| O15212 | PFD6_HUMAN | Prefoldin subunit 6 | PFDN6 |
| P52926 | HMGA2_HUMAN | High mobility group | HMGA2 |
| | | protein HMGI-C | |
| P61956 | SUMO2_HUMAN | Small ubiquitin-related | SUMO2 |
| | | modifier 2 | |
| Q9UHV9 | PFD2_HUMAN | Prefoldin subunit 2 | PFDN2 |
| P20929- | NEB_HUMAN | Nebulin | NEB |
| [2, 3] | | | |
| P09429 | HMGB1_HUMAN | High mobility group | HMGB1 |
| | | protein B1 | |
| P62328 | TYB4_HUMAN | Thymosin beta-4 | TMSB4X |
| P09497- | CLCB_HUMAN | Clathrin light chain B | CLTB |
| [2] | | | |
| P35749- | MYH11_HUMAN | Myosin-11 | MYH11 |
| [2-4] | | | |
| Q16629- | SRSF7_HUMAN | Serine/arginine-rich | SRSF7 |
| [2-4] | | splicing factor 7 | |
| P26373 | RL13_HUMAN | 60S ribosomal protein L13 | RPL13 |
| P01861 | IGHG4_HUMAN | Ig gamma-4 chain C region | IGHG4 |
| Q13200 | PSMD2_HUMAN | 26S proteasome non- | PSMD2 |
| | | ATPase regulatory | |
| | | subunit 2 | |
| P13797 | PLST_HUMAN | Plastin-3 | PLS3 |
| P49721 | PSB2_HUMAN | Proteasome subunit beta | PSMB2 |
| | | type-2 | |
| P48643 | TCPE_HUMAN | T-complex protein 1 | CCT5 |
| | | subunit epsilon | |
| P11166 | GTR1_HUMAN | Solute carrier family 2, | SLC2A1 |
| | | facilitated glucose | |
| | | transporter member 1 | |
| P61158 | ARP3_HUMAN | Actin-related protein 3 | ACTR3 |
| P09211 | GSTP1_HUMAN | Glutathione S-transferase P | GSTP1 |
| P78417 | GSTO1_HUMAN | Glutathione S-transferase | GSTO1 |
| | | omega-1 | |
| P13667 | PDIA4_HUMAN | Protein disulfide-isomerase | PDIA4 |
| | | A4 | |
| P62277 | RS13_HUMAN | 40S ribosomal protein S13 | RPS13 |
| Q13509 | TBB3_HUMAN | Tubulin beta-3 chain | TUBB3 |
| P30048 | PRDX3_HUMAN | Thioredoxin-dependent | PRDX3 |
| | | peroxide reductase, | |
| | | mitochondrial | |
| Q01813- | PFKAP_HUMAN | ATP-dependent 6- | PFKP |
| [2] | | phosphofructokinase, | |
| | | platelet type | |
| P50213 | IDH3A_HUMAN | Isocitrate dehydrogenase | IDH3A |
| | | [NAD] subunit alpha, | |
| | | mitochondrial | |
| Q9Y6N5 | SQRD_HUMAN | Sulfide:quinone oxido- | SQRDL |
| | | reductase, mitochondrial | |
| P38606- | VATA_HUMAN | V-type proton ATPase | ATP6V1A |
| [2] | | catalytic subunit A | |

In one preferred embodiment, the at least one further protein is selected from TUBB3, HMGA2 or HMGN1. In a related embodiment, the “at least one further protein” herein refers to one further protein or two further proteins.

Accordingly, in one embodiment, the first protein in context of the inventions is encoded by TUBB3, and the at least one further protein is encoded by HMGA2 or HMGN1.

In a further embodiment, the first protein in context of the inventions is encoded by TUBB3, and two at least one further proteins are encoded by HMGA2 and HMGN1.

Accordingly, in one further embodiment, the first protein in context of the inventions is encoded by HMGA2, and the at least one further protein is encoded by TUBB3 or HMGN1.

Accordingly, in one further embodiment, the first protein in context of the inventions is encoded by HMGN1, and the at least one further protein is encoded by TUBB3 or HMGA2.

It will be understood by the skilled in the art that in context of the present inventions when the first protein is encoded by TUBB3 the at least one further protein is encoded by a gene selected from the list of genes excluding TUBB 3, i.e. the at least one further protein is encoded by a gene selected from the group of genes consisting of HMGN1, HIST1H2BK, PPFIA2, COXSA, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7, HMGA2, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13.

In analogy, when the first protein is encoded by HMGA2 the at least one further protein is encoded by a gene selected from the list of genes excluding HMGA2, i.e. the at least one further protein is encoded by a gene selected from the group of genes consisting of HMGN1, HIST1H2BK, PPFIA2, COXSA, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7, TUBB3, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13.

Furthermore, when the first protein is encoded by HMGN1 the at least one further protein is encoded by a gene selected from the list of genes excluding HMGN1, i.e. the at least one further protein is encoded by a gene selected from the group of genes consisting of HIST1H2BK, PPFIA2, COXSA, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7, TUBB3, HMGA2, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13.

“Expression level” herein refers to the protein level of the gene product and may be referred to as a value.

As used herein, the term “determining” includes qualitative and/or quantitative detection (i.e. detecting and/or measuring the expression level) with or without reference to a control or a predetermined expression level.

As used herein, “detecting” means determining if the biomarker, i.e. the protein encoded by a gene as defined herein above, is present or not in a biological sample and “measuring” means determining the amount of said biomarker, i.e. the amount of the protein encoded by a gene as defined herein above in a skin sample.

The expression level of the first protein or the at least one further protein may be determined by detecting the translation product(s) (i.e. the proteins) of the genes as defined in the methods of the invention using immunologic methods using any capture ligand that is capable of binding the protein of interest. A capture ligand may be selected from the group constituted of an antibody, an aptamer, and a polypeptide which specifically recognises the amino acid sequence of interest, typically the capture ligand is a polyclonal or monoclonal antibody. Accordingly suitable immunologic methods include immuno-histochemistry (IHC), enzyme linked immunoassays (ELISA), sandwich, direct, indirect, or competitive ELISA assays, enzyme linked immunospotassays (ELIspot), radio immunoassays (RIA), flow-cytometry assays (FACS), Western Blot, fluorescence resonance energy transfer (FRET) assays, protein chip assays using for example antibodies, antibody fragments, receptor ligands or other agents binding to the proteins encoded by the genes as defined in context of the present invention.

In some embodiment, the expression level can also be determined using biophysical chemistry methods such as Mass Spectrometry.

As known by the skilled in the art antibodies that may be used in suitable immunologic methods are commercially available.

For example, the expression level of a first protein encoded by TUBB3 may be determined using a suitable immunologic method such as Western Blot using the beta-3 Tubulin Antibody (2G10) MA1-118 as available from Thermofisher or Invitrogen.

For example, the expression level of a first protein encoded by HMGA2 may be determined using a suitable immunologic method such as Western Blot using the HMGA2 Antibody PA5-25276 as available from, for example, Invitrogen.

For example, the expression level of a first protein encoded by HMGN1 may be determined using a suitable immunologic method such as Western Blot using the HMGN1 Antibody ab5212 as available from, par example, Abcam.

In one embodiment, the expression level of the first protein and the at least one further protein is determined using an enzyme linked immunoassay (ELISA) or Western Blot.

It will be understood by the skilled in the art that the expression level of a first protein encoded by a specific gene as well as the expression level of the at least one further protein encoded by a specific gene herein refers to all isoforms encoded by said gene.

It will be further understood by the skilled in the art, that the amount of the first protein and the at least one further protein as determined in a skin sample in step a) or b) of the methods of the invention depends on the amount, quality and the representativity of the cells contained in the skin sample used.

Accordingly, in one embodiment, it will therefore be understood by the skilled in the art that the expression level of the first protein and the at least one further protein in step a) typically refer to a normalized expression level.

“Normalization” herein refers to scaling data in such a way that different data sets obtained, for example, for different samples, can be compared. In one example, normalization may be performed using the total amount of proteins measured in said sample. However, normalization may also be performed by other methods known to the skilled in the art, for example by using the amount of protein encoded by so-called reference gene(s) or housekeeping gene(s).

Accordingly, in one embodiment, the “expression level” as referred to in context of the present invention is a normalized expression level.

In one embodiment, the expression level is normalized using the total amount of proteins determined in the skin sample.

Methods to determine the total amount of proteins are known to the skilled in the art. In one example, the total amount of protein of the skin sample is determined using BCA protein assay kit (Thermo Fisher Scientific, IL, USA).

The skin sample in context of the present invention is as defined herein above in the section “definitions”.

The methods of the invention further comprise a step of comparing the expression level of said first protein with a reference level and comparing the expression level of the at least one further protein with a reference level.

In one embodiment, the “reference expression level” in context of the inventions refers to a reference expression level of a protein encoded by the same gene in a skin sample of a subject, preferably of a younger subject, more preferably of a young subject.

Preferably the reference expression level is measured in a skin sample obtained from the same skin region and obtained with the same method as the skin sample of the subject of step a) of the methods of the invention.

In one example those reference expression level are predetermined reference expression level and can be a specific value or a range.

The reference expression level can be any number of statistical measures to distinguish, for instance, between a level of expression of specific gene indicative for younger skin, preferably young skin.

Accordingly, in some embodiments, the reference expression level is the protein expression level of a specific gene in a skin sample obtained from a subject, preferably from a younger subject, more preferably from a young subject.

Accordingly, in some embodiments, the reference expression level is the median protein expression level of a specific gene in skin sample obtained from a subject, preferably a younger subject, more preferably a young subject, or a population of subjects, preferably a population of younger subjects, more preferably a population of young subjects.

In one embodiment, the reference expression level is a threshold value as determined by a receiver operating characteristic (ROC) curve.

In one embodiment, a reference expression level may be determined using at least 2, 4, 10, 30, 50 or 100 samples, said samples being preferably from different subjects.

In one embodiment, a reference expression level may be determined using at least two samples, said samples being preferably from different subjects.

The terms “younger”, “young”, “old”, and “older” herein refer to the chronological age and “younger” for example refers to the chronological age of the subject from who the reference sample derived in comparison to the chronological age of the subject to which the methods of the invention are applied, i.e. the subject in which it will be determined if the skin of a subject presents signs of physiological aging.

“Skin aging” as defined herein above usually starts with a chronological age of 32 without considering extrinsic aging factors that accelerate aging.

Accordingly, in one embodiment, “young” herein refers to an age of 18 to 32, preferably less than 32, more preferably less than 30.

In one embodiment, “Old” refers to an age of 40 to 65 or above, preferably more than 40, more than 45, more than 50, more than 55, more preferably more than 65.

It will be understood by the skilled in the art that, when an expression level is compared with a reference expression level, said expression level is either higher or lower than the reference expression level. Preferably, when the expression level of a protein in context of the present invention is higher than the reference expression level, its level is significantly higher than the reference expression level. Preferably, when the expression level of a protein in context of the present invention is lower than the reference expression level, its level is significantly lower than the reference expression level.

“Significantly” herein refers to statistically significant.

In one embodiment, a p<0.05 value is considered statistically significant.

In one embodiment, the expression level of the first protein and the expression level of the at least one further protein are compared with their reference expression levels using a ratio, wherein the expression level as measured in step a) or b) of the methods of the inventions is divided through the reference expression level, if not defined otherwise.

Accordingly, in one embodiment, when a ratio of expression in context of the present invention is higher than 1, the ratio of expression is significantly higher than 1, such as 1.1, 1.2, 1.3, 1.4, 1.5, 1.6, 1.8, 2, 2.2, 2.3, 2.4, 2.6, 2.8, 3.0, 3.2, 3.4.

It will be further understood by the skilled in the art, that a ratio of expression that is higher than 1 indicates that a protein is upregulated (overexpressed) in comparison to the reference expression level, when said ratio is obtained by dividing said protein expression level through a reference expression level of the same protein.

In a related embodiment, when a ratio of expression in context of the present invention is lower than 1, the ratio of expression is significantly lower than 1, such as 0.9, 0.8, 0.7, 0.6, 0.5, 0.4, 0.3, 0.2, 0.1.

It will be understood by the skilled in the art, that a ratio of expression that is lower than 1 indicates that a protein is downregulated in comparison to the reference expression level, when said ratio is obtained by dividing said protein expression level through a reference expression level of the same protein.

### Method to Determine if the Skin of a Subject Presents Signs of Physiological Skin Aging

In one embodiment, the in vitro method to determine if the skin of a subject presents signs of physiological skin aging further comprises a step of comparing the expression level of said first protein with a reference level and comparing the expression level of the at least one further protein with a reference level.

Accordingly, in one embodiment, the in vitro method to determine if the skin of a subject presents signs of physiological skin aging comprises the steps of

a) determining in a skin sample of said subject the expression level of a first protein encoded by a gene selected from the group of genes consisting of TUBB3, HMGA2 and HMGN1, preferably TUBB3 and HMGA2, more preferably TUBB3, and the expression level of at least one further protein encoded by a gene selected from the group of genes consisting of HMGN1, HIST1H2BK, PPFIA2, COX5A, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7, TUBB3, HMGA2, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13,

b) comparing the expression level of said first protein with a reference level and comparing the expression level of the at least one further protein with a reference level, and

c) determining if the skin presents signs of physiological skin aging.

The reference level is as defined herein above in the section “methods of the invention”.

In one particular embodiment, the expression level of said first protein is compared in step b) with a reference level by determining a ratio of expression of said first protein by dividing the expression level of said first protein obtained in step a) through a reference expression level, and

the expression level of the at least one further protein is compared with a reference level in step b) by determining a ratio of expression of said at least one further protein by dividing the expression level of said at least one further protein obtained in step a) through a reference expression level.

In one embodiment, the expression level of said first protein is compared with a reference level of said first protein and the expression level of the at least one further protein is compared with a reference level of the at least one further protein.

Accordingly, in one preferred embodiment, the expression level of a first protein and at least one further protein is compared with a reference expression level by determining a ratio of expression of said first protein by dividing the expression level of said first protein obtained in step a) through a reference expression level of said first protein and by determining a ratio of expression of said at least one further protein by dividing the expression level of said at least one further protein obtained in step a) through a reference expression level said at least one further protein.

Accordingly, in one further embodiment, the invention concerns an in vitro method to determine if the skin of a subject presents signs of physiological skin aging comprising the steps of

a) determining in a skin sample of said subject the expression level of a first protein encoded by a gene selected from the group of genes consisting of TUBB3, HMGA2 and HMGN1, preferably TUBB3 and HMGA2, more preferably TUBB3, and the expression level of at least one further protein encoded by a gene selected from the group of genes consisting of HMGN1, HIST1H2BK, PPFIA2, COX5A, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7, TUBB3, HMGA2, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13,

b) determining a ratio of expression of said first protein by dividing the expression level of said first protein obtained in step a) through a reference expression level of said first protein and determining a ratio of expression of said at least one further protein by dividing the expression level of said at least one further protein obtained in step a) through a reference expression level of said at least one further protein, and

c) determining if the skin presents signs of physiological skin aging.

In one embodiment, the skin of said subject presents signs of aging, when the ratio of expression determined in step b) is higher or lower than 1 for the first protein encoded by a gene selected from the group of genes consisting of TUBB3, HMGA2 and HMGN1, preferably TUBB3 and HMGA2, more preferably TUBB3, and the ratio determined in step b) is higher or lower than 1 for the at least one further protein encoded by a gene selected from the group of genes consisting of HMGN1, HIST1H2BK, PPFIA2, COX5A, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7, TUBB3, HMGA2, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13.

In a further embodiment, the skin of said subject presents signs of aging, when the ratio of expression determined in step b) is higher than 1 for the first protein encoded by a gene selected from the group of genes consisting of TUBB3 and HMGA2, preferably TUBB3, and

the ratio determined in step b) is higher than 1 for the at least one further protein encoded by a gene selected from the group of genes consisting of TUBB3, HMGA2, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13, or

the ratio determined in step b) is higher than 1 for the first protein encoded by a gene selected from the group of genes consisting of TUBB3 and HMGA2, preferably TUBB3, and

the ratio determined in step b) is less than 1 for the at least one further protein encoded by a gene selected from the group of genes consisting of HMGN1, HIST1H2BK, PPFIA2, COX5A, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7, or

the ratio determined in step b) is less than 1 for the first protein encoded by the gene HMGN1, and

the ratio determined in step b) is higher than 1 for the at least one further protein encoded by a gene selected from the group of genes consisting of TUBB3, HMGA2, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13, or

the ratio determined in step b) is less than 1 for the first protein encoded by the gene HMGN1, and

the ratio determined in step b) is less than 1 for the at least one further protein encoded by a gene selected from the group of genes consisting of HMGN1, HIST1H2BK, PPFIA2, COXSA, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7.

Accordingly, in one embodiment, the invention refers to an in vitro method to determine if the skin of a subject presents signs of physiological skin aging comprising the steps of

a) determining in a skin sample of said subject the expression level of a first protein encoded by a gene selected from the group of genes consisting of TUBB3, HMGA2 and HMGN1, preferably TUBB3 and HMGA2, more preferably TUBB3, and determining in a skin sample of said subject the expression level of at least one further protein encoded by a gene selected from the group of genes consisting of HMGN1, HIST1H2BK, PPFIA2, COXSA, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7, TUBB3, HMGA2, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13,

b) determining a ratio of expression of said first protein by dividing the expression level of said first protein obtained in step a) through a reference expression level of said first protein and determining a ratio of expression of said at least one further protein by dividing the expression level of said at least one further protein obtained in step a) through a reference expression level of said at least one further protein, and

c) determining if the skin presents signs of physiological skin aging, wherein the skin of said subject presents signs of aging, when

the ratio of expression determined in step b) is higher than 1 for the first protein encoded by a gene selected from the group of genes consisting of TUBB3 and HMGA2, preferably TUBB3, and
the ratio determined in step b) is higher than 1 for the at least one further protein encoded by a gene selected from the group of genes consisting of TUBB3, HMGA2, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13, or

the ratio determined in step b) is higher than 1 for the first protein encoded by a gene selected from the group of genes consisting of TUBB3 and HMGA2, preferably TUBB3, and

the ratio determined in step b) is less than 1 for the at least one further protein encoded by a gene selected from the group of genes consisting of HMGN1, HIST1H2BK, PPFIA2, COXSA, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7, or

the ratio determined in step b) is less than 1 for the first protein encoded by the gene HMGN1, and

the ratio determined in step b) is higher than 1 for the at least one further protein encoded by a gene selected from the group of genes consisting of TUBB3, HMGA2, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13, or

the ratio determined in step b) is less than 1 for the first protein encoded by the gene HMGN1, and

the ratio determined in step b) is less than 1 for the at least one further protein encoded by a gene selected from the group of genes consisting of HMGN1, HIST1H2BK, PPFIA2, COXSA, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7.

In one embodiment, the skin of said subject presents signs of physiological aging, when the ratio determined in step b) is higher than 1.4 for the at least one protein selected from the group constituted of TUBB3, HMGA2, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13.

In a further embodiment, skin of said subject presents signs of physiological aging, when the ratio determined in step b) is less than 0.7 for the at least one protein selected from the group constituted of

HMGN1, HIST1H2BK, PPFIA2, COXSA, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7.

### Method of Cosmetic Treatment

The present invention further refers to a method of cosmetic treatment as defined above in the section “summary of the invention”.

A cosmetic treatment herein refer to a treatment that is capable of reducing or reversing the visible signs of physiological skin aging. As explained herein above, skin aging refers to features such as wrinkles, sunspots, uneven skin color, and sagging skin which might be are influenced by intrinsic factors, such as chronological aging and extrinsic factors, such as environmental factors, or are due to a skin disease.

As it is known to the skilled in the art a smooth skin without visible signs of aging may be considered beautiful. Accordingly, in one embodiment, reducing or reversing the visible signs of physiological skin aging is performed in order to improve a person's appearance. Accordingly, in one embodiment, the present invention refers to a “cosmetic treatment” performed in order to improve a person's appearance and is thus non-therapeutic.

As mentioned herein above, physiological aging of the skin might also be induced by a skin disease as defined herein above in the section “definitions” and may be referred to as “skin disease derived skin aging”, in such a case the cosmetic method of treatment may be prescribed, for example, by a dermatologist.

Accordingly, in one particular embodiment the physiological skin aging is skin disease derived skin aging.

In one embodiment the method of cosmetic treatment and the use as defined herein above further comprises a step of comparing the expression level of said first protein with a reference level and comparing the expression level of the at least one further protein with a reference level as defined herein above in the section “Method to determine if the skin of a subject presents signs of physiological skin aging”.

The reference level is as defined herein above in the section “methods of the invention”.

Accordingly, in one embodiment, the invention refers to a method of cosmetic treatment capable of reducing or reversing the visible signs of physiological skin aging on a subject comprising the steps of

a) determining in a skin sample of said subject the expression level of a first protein encoded by a gene selected from the group of genes consisting of TUBB3, HMGA2 and HMGN1, preferably TUBB3 and HMGA2, more preferably TUBB3, and the expression level of at least one further protein encoded by a gene selected from the group of genes consisting of HMGN1, HIST1H2BK, PPFIA2, COXSA, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7, TUBB3, HMGA2, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13,

b) comparing the expression level of said first protein with a reference level and comparing the expression level of the at least one further protein with a reference level,

c) deducing from the comparison in step b) if the skin presents signs of physiological skin aging, and

d) if the skin is determined as presenting signs of physiological aging, treating said subject with a cosmetic composition that reduces or reverses the visible signs of physiological skin aging.

“A cosmetic composition that reduces or reverses the visible signs of physiological skin aging” refers to any composition known from the skilled in the art that may reduce or reverse the visible signs of physiological skin aging, such as compositions comprising antioxidants and/or re tinols and their derivatives and are for example described in Gancevicuiene R. et al. 2012 (Dermatoendocrinol. 2012 Jul. 1; 4(3):308-19).

“Antioxidants” include but are not limited to antioxidants such as vitamin C, resveratrol and polyphenols.

“Retinol” also known as “Vitamin A” is known to the skilled in the art as the biosynthesis of collagen and reducing the expression of MMP1 (collagenase 1) and thus having a positive effect on extrinsic and intrinsic skin aging.

A “derivative of retinol” is for example tretinoid.

The step b) of “comparing the expression level of said first protein with a reference level and comparing the expression level of the at least one further protein with a reference level” and the “reference expression level” is as defined herein above.

In one particular embodiment, the expression level of said first protein is compared in step b) with a reference level by determining a ratio of expression of said first protein by dividing the expression level of said first protein obtained in step a) through a reference expression level, and

the expression level of the at least one further protein is compared with a reference level in step b) by determining a ratio of expression of said at least one further protein by dividing the expression level of said at least one further protein obtained in step a) through a reference expression level.

In one embodiment, the expression level of said first protein is compared with a reference level of said first protein and the expression level of the at least one further protein is compared with a reference level of the at least one further protein.

Accordingly, in one embodiment, the invention refers to a method of cosmetic treatment capable of reducing or reversing the visible signs of physiological skin aging on a subject comprising the steps of

a) determining in a skin sample of said subject the expression level of a first protein encoded by a gene selected from the group of genes consisting of TUBB3, HMGA2 and HMGN1, preferably TUBB3 and HMGA2, more preferably TUBB3, and the expression level of at least one further protein encoded by a gene selected from the group of genes consisting of HMGN1, HIST1H2BK, PPFIA2, COXSA, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7, TUBB3, HMGA2, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13,

b) determining a ratio of expression of said first protein by dividing the expression level of said first protein obtained in step a) through a reference expression level of said first protein and determining a ratio of expression of said at least one further protein by dividing the expression level of said at least one further protein obtained in step a) through a reference expression level of said at least one further protein, and

c) deducing from the comparison in step b) if the skin presents signs of physiological skin aging, and

d) if the skin is determined as presenting signs of physiological aging, treating said subject with a cosmetic composition that reduces or reverses the visible signs of physiological skin aging.

The embodiments defining when the skin of said subject presents signs of aging are as defined herein above in the section “Method to determine if the skin of a subject presents signs of physiological skin aging” and apply mutatis mutandis to the method of cosmetic treatment.

According to the above, in one embodiment, the invention refers to a method of cosmetic treatment capable of reducing or reversing the visible signs of physiological skin aging on a subject comprising the steps of

a) determining in a skin sample of said subject the expression level of a first protein encoded by a gene selected from the group of genes consisting of TUBB3, HMGA2 and HMGN1, preferably TUBB3 and HMGA2, more preferably TUBB3, and the expression level of at least one further protein encoded by a gene selected from the group of genes consisting of HMGN1, HIST1H2BK, PPFIA2, COXSA, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7, TUBB3, HMGA2, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13,

b) determining a ratio of expression of said first protein by dividing the expression level of said first protein obtained in step a) through a reference expression level of said first protein and determining a ratio of expression of said at least one further protein by dividing the expression level of said at least one further protein obtained in step a) through a reference expression level of said at least one further protein, and

c) deducing from the ratio obtained in step b) if the skin presents signs of physiological skin aging,

wherein the skin presents signs of physiological skin aging if
the ratio determined in step b) is higher than 1 for the first protein encoded by a gene selected from the group of genes consisting of TUBB3 and HMGA2, preferably TUBB3, and
the ratio determined in step b) is higher than 1 for the at least one further protein encoded by a gene selected from the group of genes consisting of TUBB3, HMGA2, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13, or
the ratio determined in step b) is higher than 1 for the first protein encoded by a gene selected from the group of genes consisting of TUBB3 and HMGA2, preferably TUBB3, and
the ratio determined in step b) is less than 1 for the at least one further protein encoded by a gene selected from the group of genes consisting of HMGN1, HIST1H2BK, PPFIA2, COX5A, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7, or
the ratio determined in step b) is less than 1 for the first protein encoded by the gene HMGN1, and
the ratio determined in step b) is higher than 1 for the at least one further protein encoded by a gene selected from the group of genes consisting of TUBB3, HMGA2, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13, or
the ratio determined in step b) is less than 1 for the first protein encoded by the gene HMGN1, and
the ratio determined in step b) is less than 1 for the at least one further protein encoded by a gene selected from the group of genes consisting of HMGN1, HIST1H2BK, PPFIA2, COX5A, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7, and

d) if the skin is determined as presenting signs of physiological aging, treating said subject with a cosmetic composition that reduces or reverses the visible signs of physiological skin aging.

In one embodiment, the skin of said subject presents signs of physiological aging if the ratio determined in step b) is higher than 1.4 for the at least one protein selected from the group constituted of TUBB3, HMGA2, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13.

In a further embodiment, skin of said subject presents signs of physiological aging if the ratio determined in step b) is less than 0.7 for the at least one protein selected from the group constituted of

HMGN1, HIST1H2BK, PPFIA2, COX5A, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7.

### Method to Identify a Substance

The inventors demonstrated that upon aging the expression of different proteins changes and is up-/ or downregulated in comparison to the expression of the same protein in younger skin.

Accordingly, a substance that is capable of reducing or reversing the visible signs of physiological skin aging is able of either recalibrating these differences in protein expression or reduce said up-/ or downregulated of the some of those proteins in comparison to younger skin.

“Treating a skin sample with a candidate substance” in step a) herein refers for example to contacting the skin sample, preferably the skin sample cells, with a candidature substance for from 1 min to 48 hours, preferably for 1 to 30 hours, 1 to 24 hours, 2 to 24 hours, 4 to 24 hours, 6 to 24 hours, 7 to 24 hours, 8 to 24 hours, 8 to 12 hours, such as 2, 4, 6, 8, 10, 12, 14, 16, 18, 20, 24 hours.

The skilled in the art knows that the time to contact the skin sample, preferably the skin sample cells, with a candidature substance depends on factors such as the concentration of the candidate substance and the culture condition of the skin sample, in particular the skin sample cells.

In one embodiment the skin sample is contacted with the candidate substance for at least 5 min, at least 10 min, at least 20 min, at least 40 min, at least 1 hrs, at least 2 hrs, at least 3 hrs, at least 4 hrs, at least 5 hrs, at least 6 hrs, at least 7 hrs, at least 8 hrs.

“Determining in said skin sample the expression level” in step b) herein refers for example to determine the expression level, as defined herein above, at least 6 hrs, at least 8 hrs, at least 10 hrs, at least 12 hrs, at least 14 hrs, at least 18 hrs, at least 24 hrs after contacting the skin sample with the candidature substance.

It will be understood by the skilled in the art that the word “after” in “after contacting the skin sample with the candidature substance” refers to the time point when a skin sample and the substance are put into contact. For example, if the expression is determined 8 hrs after contacting the skin sample with the candidature substance and the substance is contacted with the cells for 6 hrs, the expression will be determined in step b) 2 hrs after the 6 hrs contact time of step a).

It will be understood by the skilled in the art that, preferably, the skin sample of step a) treated with a candidate substance and the skin sample of step c) that has not been treated with a candidate substance are identical prior to step a). “Identical” herein refers to a skin sample that has been obtained by typically one biopsy wherein the sample and the cells comprised therein have undergone the same treatment, including eventually freezing of the sample and eventually undergone a cell isolation and cell culture. Typically, a skin sample, in particular the skin sample cells, will be divided into two parts prior to step a) of the method of the invention.

It will be further understood by the skilled in the art that, preferably, the skin sample in context of the method to identify a substance is reconstructed skin, preferably 3D reconstructed skin.

In one embodiment, the skin sample of step a) treated with a candidate substance and the skin sample of step c) that has not been treated with a candidate substance are further exposed to an environment that induces skin aging.

In a related embodiment, the method of identifying a candidate substance further comprises a step a2) of exposing the skin sample to an environment that induces skin aging. In the same embodiment, the skin sample that has not been treated with said candidate substance is exposed to the same environment that induces skin aging as the skin sample of step a).

“Environment that induces skin aging” herein refers for example to the exposition to oxidative stress inducers, pro-inflammatory cytokines, pollutants and UV irradiation, more particularly UV-A irradiation.

“Pro-inflammatory cytokines” are known to the skilled in the art and include, but are not limited to IL1β and TNF.

“The visible signs of physiological skin aging” are as defined herein above.

In one particular embodiment, the expression level of said first protein and said at least one further protein as determined in step b) is compared in step c) with the expression level of said first protein and said at least one further protein in a skin sample that has not been treated with said candidate substance by determining a ratio of expression of said first protein by dividing the expression level of the first protein determined in a skin sample that has not been treated with the candidate substance in step c) through the expression level of the first protein obtained in step b) and by determining a ratio of expression of said at least one further protein by dividing the expression level of the at least one further protein obtained in step c) through the expression level obtained in step b).

Accordingly, in one embodiment the invention refers to a method to identify a substance that is capable of reducing or reversing the visible signs of physiological skin aging comprising the steps of

a) treating a skin sample with a candidate substance,

b) determining in skin sample the expression level of a first protein encoded by a gene selected from the group of genes consisting of TUBB3, HMGA2 and HMGN1, preferably TUBB3 and HMGA2, more preferably TUBB3, and the expression level of at least one further protein encoded by a gene selected from the group of genes consisting of HMGN1, HIST1H2BK, PPFIA2, COX5A, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7, TUBB3, HMGA2, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13,

c) determining a ratio of expression of said first protein by dividing the expression level of said first protein determined in a skin sample that has not been treated through the expression level of said first protein obtained in step b) and determining a ratio of expression of said at least one further protein by dividing the expression level of said at least one further protein determined in a skin sample that has not been treated through the expression level said least one further protein obtained in step b),

d) identifying the candidate substance as a substance that reduces or reverses the visible signs of physiological skin aging.

In one embodiment, a substance is identified in step d) as being capable of reducing or reversing the visible signs of physiological skin aging, if the ratio of expression determined in step c) is higher or lower than 1 for the first protein encoded by a gene selected from the group of genes consisting of TUBB3, HMGA2 and HMGN1, preferably TUBB3 and HMGA2, more preferably TUBB3, and

the ratio determined in step c) is higher or lower than 1 for the at least one further protein encoded by a gene selected from the group of genes consisting of HMGN1, HIST1H2BK, PPFIA2, COX5A, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7, TUBB3, HMGA2, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13.

In a further embodiment, a substance is identified in step d) as being capable of reducing or reversing the visible signs of physiological skin aging, if the ratio of expression determined in step c) is higher than 1 for the first protein encoded by a gene selected from the group of genes consisting of TUBB3 and HMGA2, preferably TUBB3, and

the ratio determined in step c) is higher than 1 for the at least one further protein encoded by a gene selected from the group of genes consisting of TUBB3, HMGA2, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13, or

the ratio determined in step c) is higher than 1 for the first protein encoded by a gene selected from the group of genes consisting of TUBB3 and HMGA2, preferably TUBB3, and

the ratio determined in step c) is less than 1 for the at least one further protein encoded by a gene selected from the group of genes consisting of HMGN1, HIST1H2BK, PPFIA2, COXSA, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7, or

the ratio determined in step c) is less than 1 for the first protein encoded by the gene HMGN1, and

the ratio determined in step c) is higher than 1 for the at least one further protein encoded by a gene selected from the group of genes consisting of TUBB3, HMGA2, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13, or

the ratio determined in step c) is less than 1 for the first protein encoded by the gene HMGN1, and

the ratio determined in step c) is less than 1 for the at least one further protein encoded by a gene selected from the group of genes consisting of HMGN1, HIST1H2BK, PPFIA2, COXSA, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7.

Accordingly, in one particular embodiment, the invention refers to a method to identify a substance that is capable of reducing or reversing the visible signs of physiological skin aging comprising the steps of

a) treating a skin sample with a candidate substance,

b) determining in said skin sample the expression level of a first protein encoded by a gene selected from the group of genes consisting of TUBB3, HMGA2 and HMGN1, preferably TUBB3 and HMGA2, more preferably TUBB3, and the expression level of at least one further protein encoded by a gene selected from the group of genes consisting of HMGN1, HIST1H2BK, PPFIA2, COX5A, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7, TUBB3, HMGA2, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13,

c) determining a ratio of expression of said first protein by dividing the expression level of said first protein determined in a skin sample that has not been treated through the expression level of said first protein obtained in step b) and determining a ratio of expression of said at least one further protein by dividing the expression level of said at least one further protein determined in a skin sample that has not been treated through the expression level said least one further protein obtained in step b),

d) identifying the candidate substance as a substance that reduces or reverses the visible signs of physiological skin aging, if

the ratio determined in step c) is higher than 1 for the first protein encoded by a gene selected from the group of genes consisting of TUBB3 and HMGA2, preferably TUBB3, and

the ratio determined in step c) is higher than 1 for the at least one further protein encoded by a gene selected from the group of genes consisting of TUBB3, HMGA2, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13, or

the ratio determined in step c) is higher than 1 for the first protein encoded by a gene selected from the group of genes consisting of TUBB3 and HMGA2, preferably TUBB3, and

the ratio determined in step c) is less than 1 for the at least one further protein encoded by a gene selected from the group of genes consisting of HMGN1, HIST1H2BK, PPFIA2, COX5A, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7, or

the ratio determined in step c) is less than 1 for the first protein encoded by the gene HMGN1, and

the ratio determined in step c) is higher than 1 for the at least one further protein encoded by a gene selected from the group of genes consisting of TUBB3, HMGA2, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13, or

the ratio determined in step c) is less than 1 for the first protein encoded by the gene HMGN1, and

the ratio determined in step c) is less than 1 for the at least one further protein encoded by a gene selected from the group of genes consisting of HMGN1, HIST1H2BK, PPFIA2, COX5A, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7.

In a further embodiment, a substance is identified in step d) as being capable of reducing or reversing the visible signs of physiological skin aging, if the ratio determined in step c) is higher than 1.4 for the at least one protein selected from the group constituted of TUBB3, HMGA2, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13.

In a further embodiment, a substance is identified in step d) as being capable of reducing or reversing the visible signs of physiological skin aging, if the ratio determined in step c) is less than 0.7 for the at least one protein selected from the group constituted of

HMGN1, HIST1H2BK, PPFIA2, COXSA, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7.

### Kit

“Capture ligand” means a ligand capable of binding a first protein encoded by a gene selected from the group of genes consisting of TUBB3, HMGA2 and HMGN1, preferably TUBB3 and HMGA2, more preferably TUBB3, or a ligand capable of binding at least one further protein encoded by a gene selected from the group of genes consisting of HMGN1, HIST1H2BK, PPFIA2, COXSA, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7, TUBB3, HMGA2, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13.

Accordingly, the capture ligand specifically binds to the amino acid sequence of the proteins encoded by a gene as referenced herein above. “Specifically binds to the amino acid sequence encoded by one of the genes listed herein above” means typically specifically binding to an epitope of said amino acid sequence.

Preferably, the capture ligand is selected from the group constituted of an antibody, an aptamer, and a polypeptide which specifically recognizes the amino acid sequence of a protein encoded by one of the genes listed herein above.

In this context, the term “antibody” refers to any polyclonal or monoclonal antibody.

The fragments scFv, Fab, Fab′, F(ab′)₂, as well as camelids single-chain antibodies are examples of antibody fragments which specifically recognizes the amino acid sequence of a protein encoded by one of the genes listed herein above.

The “aptamers” are well-known by the one skilled in the art. Aptamers are compounds of a nucleotide, in particular a ribonucleotide or desoxyribonucleotide, or a peptide nature able to bind specifically to a target, in particular a protein target. The aptamers of a nucleotide nature and the production thereof are described, in particular, by Ellington et al. (1990) Nature 346:818-22 and Bock et al. (1992) Nature 355:564-6. The aptamers of a peptide nature and the production thereof are described, in particular, by Hoppe-Seyler et al. (2000) J. Mol Med. 78:426-30.

The ligands may also be obtained by chemical synthesis or by genetic engineering.

In one preferred embodiment, the capture ligand is an antibody.

Antibodies directed against the amino acid sequence of a protein encoded by one of the genes listed herein above are commercially available.

For example, an antibody directed against a protein encoded by TUBB3 is the beta-3 Tubulin Antibody (2G10) MA1-118 as available from Thermofisher or Invitrogen.

For example, an antibody directed against a protein encoded by HMGA2 is the HMGA2 Antibody PA5-25276 as available from, for example, Invitrogen.

For example, an antibody directed against a protein encoded by HMGN1 is the HMGN1 Antibody ab5212 as available from, par example, Abcam.

The “at least one capture ligand” in context of the invention refers to one capture ligand, two capture ligands, three capture ligands, four capture ligand, five capture ligand, six further capture ligands, seven capture ligands, eight capture ligands, nine capture ligands or ten capture ligands, preferably to one capture ligand, two capture ligands, three capture ligands, four capture ligands.

The “at least one further protein” is as defined herein above.

In one particular embodiment, the kit comprises
at least one capture ligand for determining the expression level of a first protein encoded by a gene selected from the group consisting of TUBB3, HMGA2, and HMGN1, preferably TUBB3 and HMGA2, more preferably TUBB3 and
at least one capture ligand for determining the expression level of at least one further protein encoded by a gene selected from the group of genes consisting of HMGN1, HIST1H2BK, PPFIA2, COXSA, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7, TUBB3, HMGA2, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13.

In some particular embodiments, the kit comprises
at least one capture ligand for determining the expression level of one further protein encoded by TUBB3, and
at least one capture ligand for determining the expression level of one further protein encoded by HMGA2, and/or
at least one capture ligand for determining the expression level of one further protein encoded by HMGN1.

In one embodiment, the capture ligand is immobilised on a solid phase.

By way of non-limiting examples of solid phase, microplates could be used, in particular polystyrene microplates, such as those sold by Nunc, Denmark. Solid particles or beads, paramagnetic beads, such as those produced by Dynal, Merck-Eurolab (France) (under the trademark Estapor™) and Polymer Laboratories, or even polystyrene or polypropylene test tubes, glass, plastic or silicon chips, etc. may also be used.

In one embodiment, these kits may additionally comprise other components such as e.g. reagents and/or instructions.

Throughout the instant application, the term “and/or” is a grammatical conjunction that is to be interpreted as encompassing that one or more of the cases it connects may occur. For example, the wording “qualitative and/or quantitative detection” in the phrase “the term “determining” includes qualitative and/or quantitative detection” indicates that the term determining may refer to qualitative detection, or to quantitative detection, or to qualitative detection and to quantitative detection.

As used in this specification and the appended claims, the singular forms “a,” “an” and “the” include plural references, such as a plurality of the object referred to, unless the content clearly dictates otherwise.

Throughout the instant application, the term “comprising” is to be interpreted as encompassing all specifically mentioned features as well optional, additional, unspecified ones. As used herein, the use of the term “comprising” also discloses the embodiment wherein no features other than the specifically mentioned features are present (i.e. “consisting of”).

The invention will now be described in more detail with reference to the following examples. All literature and patent documents cited herein are hereby incorporated by reference. While the invention has been illustrated and described in detail in the foregoing description, the examples are to be considered illustrative or exemplary and not restrictive.

### FIGURES

FIG. 1: Determination of TUBB3 expression in elderly and young individuals using Western Blot analysis using the beta-3 Tubulin Antibody (2G10) MA1-118 as available from Thermofisher or Invitrogen. A) Western Blot of TUBB3 expression in elderly and young individuals using beta-3 Tubulin Antibody (2G10) MA1-118 B) Total Protein content as visualized on a SDS page C) Graph demonstrating the normalized amount of the protein encoded by TUBB3. Normalization was performed using the total protein concentration.

FIG. 2: Determination of TUBB3 expression in elderly and young individuals using Western Blot analysis using the beta-3 Tubulin Antibody (2G10) MA1-118 as available from Thermofisher or Invitrogen. D) Graph demonstrating the normalized amount of beta-3 Tubulin in young and elderly patients. E) Corresponding receiver operating characteristic (ROC) curve (GraphPad Prism version 7.00 for Windows, La Jolla Calif. USA, www.graphpad.com) with an Area under the curve (AUC) of 0.9048.

FIG. 3: Determination of HMGA2 expression in elderly and young individuals using Western Blot analysis using the HMGA2 Antibody PA5-25276. A) Western Blot of HMGA2 expression in elderly and young individuals using using the HMGA2 Antibody PA5-25276 B) Total Protein content as visualized on a SDS page C) Graph demonstrating the normalized amount of the protein encoded by HMGA2. Normalization was performed using the total protein concentration.

FIG. 4: Determination of HMGA2 expression in elderly and young individuals using Western Blot analysis using the HMGA2 Antibody PA5-25276. D) Graph demonstrating the normalized amount of HMGA2 in young and elderly patients. E) Corresponding receiver operating characteristic (ROC) curve (GraphPad Prism version 7.00 for Windows, La Jolla Calif. USA, www.graphpad.com) with an Area under the curve (AUC) of 0.8776.

FIG. 5: A) Receiver operating characteristic (ROC) curve (GraphPad Prism version 7.00 for Windows, La Jolla Calif. USA, www.graphpad.com) corresponding to expression of beta-tubulin in young and elderly patients, with an Area under the curve (AUC) of 0.806. B) Receiver operating characteristic (ROC) curve (GraphPad Prism version 7.00 for Windows, La Jolla Calif. USA, www.graphpad.com) corresponding to expression of HMGA2 in young and elderly patients, with an Area under the curve (AUC) of 0.939. C) Receiver operating characteristic (ROC) curve (GraphPad Prism version 7.00 for Windows, La Jolla Calif. USA, www.graphpad.com) corresponding to expression of HMGN1 in young and elderly patients, with an Area under the curve (AUC) of 0.592.

FIG. 6: Receiver operating characteristic (ROC) curve (GraphPad Prism version 7.00 for Windows, La Jolla Calif. USA, www.graphpad.com) corresponding to the combination of expressions of beta-tubulin, HMGA2 and HMGN1 in young and elderly patients, with an Area under the curve (AUC) of 1. The model equation is as follows:

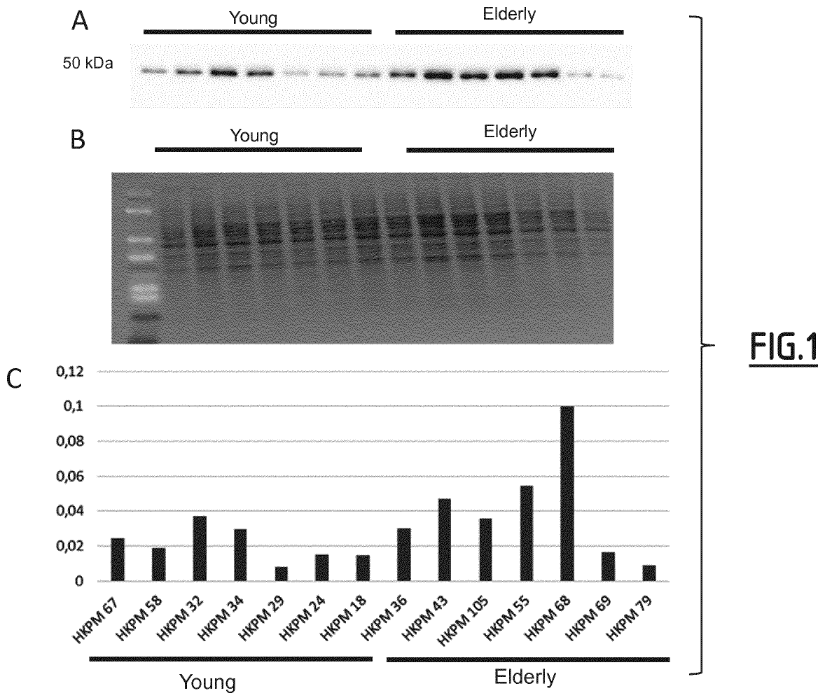

Prediction=83.76−(992.91×BETA TUBULINE)−(4936.61×HMGA2)+(273.40×HMGN1)

### EXAMPLES

### Example 1

### 1. Material and Methods

### 1.1 Cell Culture

Isolation and culture of primary keratinocytes: Skin biopsies were obtained after plastic mammary surgery following healthy person's written consent. Donors were European Caucasian women aged of 60 and 65 years (n=2) and 27 and 32 years (n=2) classified in two age groups designed hereafter elderly and young respectively (agreement No. DC-2008-444 from Codecoh (Conservation D'Eléments du Corps Humain)). Skin biopsies were sun protected non-exposed skin. Human primary keratinocytes were cultured in KSFM medium supplemented with 25 μg/mL BPE and 0.9 ng/mL EGF.

### 1.2 Protein Extraction

Frozen cells pellets were lysed for 30 minutes at 4° in a solution containing 40 mM HEPES ph 7.4, 100 mM NaCl, 1 mM EDTA, 0.02% Triton, 0.02% Sodium Deoxycholate, 0.2 mM TCEP, and protease and phosphatase inhibitor cocktail (PhosSTOP) from Roche. Lysis was achieved by short sonication on ice and the lysates were cleared by centrifugation at 14,000 rpm for 20 minutes at 4°. The concentration of the protein extract was determined using BCA protein assay kit (Thermo Fisher Scientific, IL, USA).

1.3 Protein Digestion and iTRAQ Labeling

Protein samples were labeled with iTRAQ reagents in a 8-plex set according to the manufacturer's instructions (iTRAQ Reagents 8 plex Applications kit; AB Sciex, Framingham, Mass., USA). Briefly, equal amount of protein extract obtained from cells originated from young donors were pooled in order to achieve a total of 100 rig. The same procedure was applied for cells from elderly donors. The samples were reduced in 20 mM of TCEP (tris-(2-carboxyethyl)phosphine) at 37° C. for 1 h, cysteine-residues were blocked in 10 mM of MMTS (methyl methanethiosulfonate) at room temperature for 10 min, followed by trypsin (Promega) digestion at a ratio of 1:10 (trypsin:protein) at 37° C. overnight. Each peptide solution was labeled with one iTRAQ reagent:iTRAQ reporter ions of m/z 113.1 for young and m/z 117.1 for elderly. iTRAQ labeling was verified for all reaction and the samples were pooled in a ration 1:1 and dried by vacuum centrifugation prior to the OFFGEL peptides fractionation.

### 1.4 Peptide OFFGEL Isoelectrofocusing

Peptide fractionation according to their pI was performed with 3100 OFFGEL Fractionator and the OFFGEL Kit linear pH 3-10 (Agilent Technology) in a 24-well setup following the manufacturer's instructions. The device was set up for the 24 fractions separation by using 24-cm-long IPG gel strip with a linear pH gradient ranging at 3-10. iTRAQ labeled peptide mix was dried by vacuum centrifugation and resuspended in focusing OFFGEL buffer prior loading in each of the 24 wells. Peptides were focused with a constant current of 50 μA until 50 kVh was reached. After complete fractionation, peptides samples were recovered from each well, dried in a vacuum concentrator and then desalted using C18 ZipTips (Millipore, MA, USA).

### 1.5 Reversed Phase Nano-Liquid Chromatography

Further peptide separation was performed on an Ultimate 3000 C18 reversed-phase nano liquid chromatography (RP-nanoLC) system (Ultimate 3000, Dionex/Thermo Scientific) controlled by Chromeleon v. 6.80 software (Dionex/Thermo Scientific/LC Packings, Amsterdam, The Netherlands) and coupled to a PROBOT MALDI spotting device controlled by the μCarrier 2.0 software (Dionex/Thermo Scientific/LC Packings, Amsterdam, The Netherlands).

Vacuum dried fractions were resuspended in buffer A (98% water, 2% ACN and 0.05% TFA) before injection on a nano-trapping column (C18, 3 μm, 100A pore size; LC Packing) in 2% ACN and 0.05% TFA at a flow rate of 20 μL/min for 5 min. Then, trapped peptides were separated by reversed phase chromatography (Acclaim PepMap300 75 μm, 15 cm, nanoViper C18, 3 μm, 100 Å pore size; Thermo Scientific) with a binary gradient of buffer A (2% ACN and 0.05% TFA) and buffer B (80% ACN and 0.04% TFA) at a flow rate of 0.3 μL/min. The entire run lasted 60 min and the nanoLC gradient was set up as follows: 5-35 min, 8-42% B; 35-40 min, 42-58% B; 40-50 min, 58-90% B and 50-60 min, 90% B. Fractions from eluted solution were collected and spotted on a MALDI sample plate (AB Sciex, Les Ulis, France) at a frequency of one spot per 15 seconds. The α-cyano-4-hydroxy-cinnamic acid matrix (HCCA, 2 mg/mL in 70% ACN and 0.1% TFA) was continuously added to the column effluent at a flow rate of 0.9 μL/min, and therefore, integrated in each spot of MALDI sample plate.

### 1.6 MALDI-TOF/TOF Analysis

MS and MS/MS analysis of nanoLC-off-line spotted peptide samples were performed using the 4800 MALDI-TOF/TOF Analyzer (AB Sciex, Les Ulis, France) controlled by the 4000 Series Explorer software v. 3.5. The mass spectrometer was operated in positive reflector mode. Each spectrum was externally calibrated using the Peptide Calibration Standard II (Bruker Daltonics, Bremen, Germany) and the peptide mass tolerance was set to 50 ppm. MS spectra were acquired in a m/z 700-4000 range. Up to 30 of the most intense ions per spot position characterized by a S/N (signal/noise) ratio higher than 40 were chosen for MS/MS analysis. Selected ions were fragmented by using CID (Collision Induced Dissociation) activation mode in order to obtain the corresponding MS/MS spectrum that is necessary to determine the sequence of these peptides and quantify them.
1.7 Analysis of iTRAQ Data
MS and MS/MS spectra were used for identification and relative quantitation by using ProteinPilot™ software v 4.0 with the Paragon™ Algorithm (AB Sciex, Les Ulis, France) and Mascot. The analysis was performed with the human database of UniProtKB release 2015_06—June, 2015/Swiss-Prot (European Bioinformatics Institute, Hinxton, UK). Concerning ProteinPilot search, the search effort was set to ‘Thorough ID’ and the False Discovery Rate Analysis (FDR) of 1% was applied. For quantification, bias and background correction was applied and only quantified proteins with at least 1 peptide at the 95% peptide confidence level were included. For Mascot search the FDR was set lower than 1% and only peptides with a score higher than 30 was considered. Data were merged at the peptide level after ProteinPilot and Mascot analysis. In order to obtain high quality in quantitative analysis, the inventors analyzed the data with the R package Isobar (Breitwieser et al., 2011, J. Proteome Res. 10, 2758-2766) which allows the determination of statistical significance of protein/peptide regulation. A normal fit was used and only proteins which ratio had a pValueRatio and a pValueSample <0.05 are then considered as significantly differentially expressed depending on age. For output of our quantitative iTRAQ results, all protein ratios were expressed as elderly over young (117:113) to present relative protein quantification ratios. A summary of the parameters applied for the mass data analysis is presented in Table 1 herein below.

TABLE 1
Summary of the parameters applied for the bioinformatic analysis

Parameters for peptides and proteins identification
Analysis with Ppilot and Mascot
Database: uniprot/Swissprot with variants
Ppilot settings : Biological modifications
Mascot settings: Precursor 50 ppm; MS/MS:
0.6 Da; Fixed modif: MMTS; variable
modif: deamidation (NQ) Oxidation (M)
Data merged at peptide level
Global FDR: 1% (protein level)
Peptide confidence >=95%
Mascot score >30
Number of validated peptides identified
# peptides identified: 57339 (with variants)
# Peptides identified by Mascot: 54513
# peptides identified by Ppilot: 13891
# peptides identified by Mascot and Ppilot: 11065
Parameters and results of the annalysis with the isobar R package
Isobar for statistical analysis using a Normal fit model
# protein identified: 517 (groups of proteins)
# proteins identified and quantified: 446
# proteins identified not quantified: 71
# Dysregulated proteins: 58
# Upregulated proteins (Elderly vs Young): 18
# Downregulated protéins (Elderly vs Young): 40

### 1.8 Gene Ontology and Pathway Analysis

Gene ontology and pathway analysis were performed using PANTHER (http://www.pantherdb.org/) (Mi et al., 2013, Nucleic Acids Res. 41, D377-D386) by importing the list of dysregulated proteins and the proteins were classifiied in one or several categories regarding PANTHER Family; Protein class; GO-Slim Molecular function, Biological Process and Cellular Component, and Pathway (data not shown).

### 1.9 Western Blot Analysis

Human primary keratinocytes was harvested and cultivated from skin biopsies of 8 young (Age: 18; 21; 24; 26; 27 (2 donors); 30; 32) and 10 elderly donors (57; 59; 60; 62 (2); 65 (2); 66; 68; 71). At early passages (2 or 3, when cells are still proliferating), cells were lysed by vortexing in RIPA Buffer (Sigma-Aldrich) containing protease inhibitors (Complete Mini protease inhibitor cocktail, Roche, Switzerland), 1 mM DTT and 100 μM PMSF. Samples were then centrifuged for 15 minutes at 14,000 rpm and the supernatants collected. Protein concentration was determined with MicroBC Assay (Interchim) and 20 μg of total protein was loaded on TGX Stain-Free™ FastCast™ 12% Acrylamide gels (Biorad). Proteins were transferred onto a nitrocellulose membrane using Trans-Blot® Turbo™ Transfert System (Biorad). Membranes were blocked with TBS-Tween 0.5% containing 5% non-fat milk. Primary antibodies were incubated at the following dilution in TBS-Tween 0.5% containing 5% non-fat milk overnight at 4° C.: 1/1000 for Tubulin beta-3 chain antibody (MA1-118; Thermoscientific) and 1/1000 for Cornifin-B antibody (PA5-26062; Thermoscientific). After washing in TBS-Tween 0.5%, membranes were incubated with HRP conjugated secondary antibodies (Amersham ECL anti-mouse and anti-rabbit IgG HRP-linked, whole antibody, GE Healthcare) for 1 hour at RT. Membranes were then washed in TBS-Tween 0.5% and blot images were acquired on Molecular Imager Gel Doc XR+ and Chemidoc XRS+ Systems (Biorad). Specific detected bands were quantified with Image LAb 2.0 Software (Biorad) and corresponding intensities were normalized with total protein content and expressed as a ratio.
Western blot results of TUBB3 and HMGA2 were illustrated by box plots and receiver operating characteristic curves (ROC curve) was created by using GraphPad Prism version 7.00 for Windows, (GraphPad Software, La Jolla Calif. USA, www.graphpad.com).

### 2. Results

### 2.1 Identification of Fifty Eight Proteins Differentially Expressed Depending on Age Status by Proteomic Analysis

In order to obtain a quantitative proteomic map of elderly and young donors derived keratinocytes cells, an iTRAQ labeling coupled with OFFGEL fractionation and off-line nanoLC/MS/MS was used as previously described (Martin-Bernabé et al., 2014, J. Proteome Res. 13, 4695-470). The bioinformatic analysis with ProteinPilot and Mascot resulted in the identification of 517 unique proteins using a 1% FDR and considering only proteins with at least 1 peptide with >95% confidence level and score >30. A statistical analysis with the isobar package and quantified 446 proteins was performed. Elderly cells were labeled with iTRAQ m/z 117 tag and young cells with iTRAQ m/z 113 tag. Thus, the ratio 117:113 (Elderly:Young) indicates the relative protein abundance between elderly and young cell samples. The complete list of identified proteins, including the UniProtKB accession number, ID, protein and gene name, peptide count, spectral count, sequence coverage, iTRAQ ratios and p-values ratio and p-values sample for elderly versus young cells are provided in supplemental data (Data not shown).

When the pValue ratio and the pValue sample were both <0.05, proteins were considered significantly differently expressed. Applying these criteria, 58 proteins significantly differentially expressed were identified depending on age status. From them, 40 were downregulated and 18 were upregulated with aging (Table 2 and Table 3).

Table 2: List of proteins significantly downregulated in elderly cells versus young cells (iTRAQ ratio 117/113). Statistically significant iTRAQ ratios (p-value ratio and p-value sample 0.05) for proteins down-regulated

TABLE 2
List of proteins significantly downregulated in elderly cells versus young cells (iTRAQ ratio 117/113). Statistically
significant iTRAQ ratios (p-value ratio and p-value sample ≤ 0.05) for proteins down-regulated

| | | | | | | Sequence | Ratio | | | |
|---|---|---|---|---|---|---|---|---|---|---|
| Accession/ | | | | | | Coverage | [Elderly/ | P Value | P Value | Log10 |
| variants | ID | Description | Gene | Count | Count | (%) | Young] | Rat | Sample | Ratio |
| O60814 | H2B1K_HUMAN | Histone H2B | HIST1H2BK | 9 | 131 | 4.76 | 0.170 | 6.27E−05 | 4.36E−19 | −0.769 |
| | | type 1-K | | | | | | | | |
| O75334- | LIPA2_HUMAN | Liprin-alpha-2 | PPFIA2 | 2 | 2 | 0.64 | 0.295 | 3.83E−02 | 5.16E−10 | −0.531 |
| [2-6] | | | | | | | | | | |
| P20674 | COX5A_HUMAN | Cytochrome | COX5A | 2 | 3 | 9.33 | 0.309 | 1.56E−02 | 2.23E−09 | −0.510 |
| | | c oxidase | | | | | | | | |
| | | subunit 5A, | | | | | | | | |
| | | mitochondrial | | | | | | | | |
| P04732 | MT1E_HUMAN | Metallothionein-1E | MT1E | 1 | 9 | 16.39 | 0.310 | 7.22E−13 | 2.40E−09 | −0.509 |
| P05204 | HMGN2_HUMAN | Non-histone | HMGN2 | 4 | 11 | 8.89 | 0.330 | 1.89E−02 | 1.50E−08 | −0.482 |
| | | chromosomal | | | | | | | | |
| | | protein | | | | | | | | |
| | | HMG-17 | | | | | | | | |
| Q15075 | EEA1_HUMAN | Early endosome | EEA1 | 2 | 3 | 0.64 | 0.331 | 1.71E−02 | 1.61E−08 | −0.481 |
| | | antigen 1 | | | | | | | | |
| Q9UKY7- | CDV3_HUMAN | Protein CDV3 | CDV3 | 3 | 16 | 11.63 | 0.342 | 1.18E−03 | 4.11E−08 | −0.466 |
| [2] | | homolog | | | | | | | | |
| O75152 | ZC11A_HUMAN | Zinc finger CCCH | ZC3H11A | 1 | 1 | 1.48 | 0.349 | 2.87E−02 | 7.18E−08 | −0.457 |
| | | domain-containing | | | | | | | | |
| | | protein 11A | | | | | | | | |
| P17096 | HMGA1_HUMAN | High mobility | HMGA1 | 1 | 19 | 7.48 | 0.352 | 1.56E−02 | 8.83E−08 | −0.454 |
| | | group protein | | | | | | | | |
| | | HMG-I/HMG-Y | | | | | | | | |
| P06454- | PTMA_HUMAN | Prothymosin alpha | PTMA | 5 | 24 | 12.61 | 0.357 | 4.01E−02 | 1.34E−07 | −0.447 |
| [2] | | [Cleaved into: | | | | | | | | |
| | | Prothymosin | | | | | | | | |
| | | alpha, N- | | | | | | | | |
| | | terminally | | | | | | | | |
| | | processed; | | | | | | | | |
| | | Thymosin | | | | | | | | |
| | | alpha-1] | | | | | | | | |
| Q8NC51- | PAIRB_HUMAN | Plasminogen | SERBP1 | 11 | 90 | 4.66 | 0.361 | 1.56E−02 | 1.82E−07 | −0.442 |
| [3] | | activator | | | | | | | | |
| | | inhibitor | | | | | | | | |
| | | 1 RNA-binding | | | | | | | | |
| | | protein | | | | | | | | |
| Q13442 | HAP28_HUMAN | 28 kDa heat- | PDAP1 | 1 | 1 | 7.18 | 0.363 | 1.23E−02 | 2.09E−07 | −0.440 |
| | | and acid- | | | | | | | | |
| | | stable | | | | | | | | |
| | | phosphoprotein | | | | | | | | |
| P63313 | TYB10_HUMAN | Thymosin beta-10 | TMSB10 | 2 | 35 | 13.64 | 0.364 | 1.30E−02 | 2.21E−07 | −0.439 |
| O00233 | PSMD9_HUMAN | 26S proteasome | PSMD9 | 1 | 6 | 5.38 | 0.383 | 2.34E−04 | 8.33E−07 | −0.416 |
| | | non-ATPase | | | | | | | | |
| | | regulatory | | | | | | | | |
| | | subunit 9 | | | | | | | | |
| P05114 | HMGN1_HUMAN | Non-histone | HMGN1 | 3 | 13 | 8.00 | 0.384 | 1.20E−02 | 8.80E−07 | −0.415 |
| | | chromosomal | | | | | | | | |
| | | protein | | | | | | | | |
| | | HMG-14 | | | | | | | | |
| P62158 | CALM_HUMAN | Calmodulin | CALM1 | 10 | 147 | 8.72 | 0.385 | 2.89E−02 | 9.13E−07 | −0.415 |
| P02795, | MT1G_HUMAN, | Metallothionein-1G, | MT1G, | 1 | 20 | 16.29 | 0.392 | 3.61E−03 | 1.47E−06 | −0.406 |
| P13640- | MT1X_HUMAN, | Metallothionein-1X, | MT1X, | | | | | | | |
| [2], | MT2_HUMAN | Metallothionein-2 | MT2A | | | | | | | |
| P80297 | | | | | | | | | | |
| P67936 | TPM4_HUMAN | Tropomyosin | TPM4 | 5 | 48 | 3.23 | 0.401 | 3.64E−02 | 2.51E−06 | −0.397 |
| | | alpha-4 chain | | | | | | | | |
| P22528 | SPR1B_HUMAN | Cornifin-B | SPRR1B | 4 | 56 | 8.99 | 0.405 | 2.54E−02 | 3.18E−06 | −0.392 |
| Q92538- | GBF1_HUMAN | Golgi-specific | GBF1 | 1 | 1 | 0.32 | 0.406 | 4.48E−02 | 3.34E−06 | −0.391 |
| [2, 3] | | brefeldin | | | | | | | | |
| | | A-resistance | | | | | | | | |
| | | guanine | | | | | | | | |
| | | nucleotide | | | | | | | | |
| | | exchange | | | | | | | | |
| | | factor 1 | | | | | | | | |
| P51858 | HDGF_HUMAN | Hepatoma-derived | HDGF | 4 | 12 | 3.75 | 0.429 | 2.37E−02 | 1.17E−05 | −0.368 |
| | | growth factor | | | | | | | | |
| P61604 | CH10_HUMAN | 10 kDa heat | HSPE1 | 12 | 121 | 7.84 | 0.455 | 2.20E−02 | 4.16E−05 | −0.342 |
| | | shock protein, | | | | | | | | |
| | | mitochondrial | | | | | | | | |
| P07108- | ACBP_HUMAN | Acyl-CoA-binding | DBI | 4 | 50 | 9.20 | 0.460 | 1.86E−02 | 5.21E−05 | −0.337 |
| [2-5] | | protein | | | | | | | | |
| Q9C030- | TRIM6_HUMAN | Tripartite | TRIM6 | 2 | 8 | 1.23 | 0.463 | 1.10E−02 | 6.03E−05 | −0.334 |
| [2] | | motif-containing | | | | | | | | |
| | | protein 6 | | | | | | | | |
| P20962 | PTMS_HUMAN | Parathymosin | PTMS | 4 | 11 | 8.82 | 0.468 | 1.44E−02 | 7.30E−05 | −0.330 |
| Q9GZP8 | IMUP_HUMAN | Immortalization | IMUP | 3 | 5 | 9.43 | 0.476 | 8.05E−03 | 1.05E−04 | −0.322 |
| | | up-regulated | | | | | | | | |
| | | protein | | | | | | | | |
| Q9H299 | SH3L3_HUMAN | SH3 | SH3BGRL3 | 3 | 36 | 10.75 | 0.490 | 1.62E−02 | 1.82E−04 | −0.310 |
| | | domain-binding | | | | | | | | |
| | | glutamic | | | | | | | | |
| | | acid-rich-like | | | | | | | | |
| | | protein 3 | | | | | | | | |
| P62857 | RS28_HUMAN | 40S ribosomal | RPS28 | 3 | 20 | 17.39 | 0.491 | 9.21E−03 | 1.93E−04 | −0.309 |
| | | protein | | | | | | | | |
| | | S28 | | | | | | | | |
| P16949- | STMN1_HUMAN | Stathmin | STMN1 | 3 | 24 | 8.72 | 0.492 | 2.51E−02 | 2.00E−04 | −0.308 |
| [2] | | | | | | | | | | |
| P02765 | FETUA_HUMAN | Alpha-2-HS- | AHSG | 4 | 61 | 3.27 | 0.507 | 4.77E−02 | 3.38E−04 | −0.295 |
| | | glycoprotein | | | | | | | | |
| O15212 | PFD6_HUMAN | Prefoldin subunit 6 | PFDN6 | 1 | 9 | 9.30 | 0.519 | 1.72E−02 | 5.20E−04 | −0.285 |
| P52926 | HMGA2_HUMAN | High mobility group | HMGA2 | 3 | 8 | 11.93 | | | | |
| | | protein HMGI-C | | | | | | | | |
| P61956 | SUMO2_HUMAN | Small | SUMO2 | 1 | 7 | 12.63 | 0.534 | 1.89E−02 | 8.65E−04 | −0.272 |
| | | ubiquitin-related | | | | | | | | |
| | | modifier 2 | | | | | | | | |
| Q9UHV9 | PFD2_HUMAN | Prefoldin subunit 2 | PFDN2 | 1 | 5 | 9.09 | 0.544 | 1.81E−02 | 1.16E−03 | −0.265 |
| P20929- | NEBU_HUMAN | Nebulin | NEB | 4 | 4 | 0.13 | 0.563 | 5.23E−06 | 2.03E−03 | −0.250 |
| [2, 3] | | | | | | | | | | |
| P09429 | HMGB1_HUMAN | High mobility group | HMGB1 | 5 | 33 | 5.58 | 0.563 | 9.96E−07 | 2.07E−03 | −0.249 |
| | | protein B1 | | | | | | | | |
| P62328 | TYB4_HUMAN | Thymosin beta-4 | TMSB4X | 1 | 16 | 15.91 | 0.582 | 4.60E−03 | 3.41E−03 | −0.235 |
| P09497- | CLCB_HUMAN | Clathrin light | CLTB | 7 | 24 | 3.49 | 0.593 | 1.87E−03 | 4.46E−03 | −0.227 |
| [2] | | chain B | | | | | | | | |
| P35749- | MYH11_HUMAN | Myosin-11 | MYH11 | 1 | 5 | 0.56 | 0.649 | 4.13E−02 | 1.53E−02 | −0.188 |
| [2-4] | | | | | | | | | | |
| Q16629- | SRSF7_HUMAN | Serine/arginine-rich | SRSF7 | 2 | 13 | 3.78 | 0.651 | 3.09E−02 | 1.59E−02 | −0.187 |
| [2-4] | | splicing factor 7 | | | | | | | | |

Table 3: List of proteins significantly up-regulated in elderly cells versus young cells (iTRAQ ratio 117/113). Statistically significant iTRAQ ratios (p-value ratio and p-value sample 0.05) for proteins up-regulated

TABLE 3
List of proteins significantly up-regulated in elderly cells versus young cells (iTRAQ ratio 117/113). Statistically
significant iTRAQ ratios (p-value ratio and p-value sample ≤ 0.05) for proteins up-regulated

| | | | | | | | Ratio | | | |
|---|---|---|---|---|---|---|---|---|---|---|
| Accession/ | | | | Peptide | | Coverage | [Elderly/ | P Value | P Value | Log10 |
| variants | ID | Description | Gene | Count | Count | (%) | Young] | Ratio | Sample | Ratio |
| P26373 | RL13_HUMAN | 60S ribosomal | RPL13 | 4 | 15 | 4.27 | 1.455 | 2.54E−03 | 3.04E−02 | 0.163 |
| | | protein L13 | | | | | | | | |
| P01861 | IGHG4_HUMAN | Ig gamma-4 | IGHG4 | 1 | 4 | 4.89 | 1.482 | 2.00E−02 | 2.46E−02 | 0.171 |
| | | chain | | | | | | | | |
| | | C region | | | | | | | | |
| Q13200 | PSMD2_HUMAN | 26S proteasome | PSMD2 | 1 | 5 | 1.65 | 1.511 | 4.13E−02 | 1.96E−02 | 0.179 |
| | | non-ATPase | | | | | | | | |
| | | regulatory | | | | | | | | |
| | | subunit 2 | | | | | | | | |
| P13797 | PLST_HUMAN | Plastin-3 | PLS3 | 5 | 13 | 1.90 | 1.584 | 2.13E−02 | 1.08E−02 | 0.200 |
| P49721 | PSB2_HUMAN | Proteasome | PSMB2 | 1 | 22 | 5.47 | 1.635 | 1.82E−03 | 7.01E−03 | 0.214 |
| | | subunit beta | | | | | | | | |
| | | type-2 | | | | | | | | |
| P48643 | TCPE_HUMAN | T-complex | CCT5 | 6 | 35 | 1.29 | 1.696 | 2.17E−03 | 4.15E−03 | 0.229 |
| | | protein 1 | | | | | | | | |
| | | subunit | | | | | | | | |
| | | epsilon | | | | | | | | |
| P11166 | GTR1_HUMAN | Solute carrier | SLC2A1 | 4 | 46 | 2.03 | 1.891 | 2.60E−02 | 7.28E−04 | 0.277 |
| | | family 2, | | | | | | | | |
| | | facilitated | | | | | | | | |
| | | glucose | | | | | | | | |
| | | transporter | | | | | | | | |
| | | member 1 | | | | | | | | |
| P61158 | ARP3_HUMAN | Actin-related | ACTR3 | 6 | 26 | 2.63 | 2.040 | 3.21E−02 | 1.84E−04 | 0.310 |
| | | protein 3 | | | | | | | | |
| P09211 | GSTP1_HUMAN | Glutathione | GSTP1 | 10 | 117 | 7.62 | 2.308 | 3.65E−02 | 1.47E−05 | 0.363 |
| | | S-transferase P | | | | | | | | |
| P78417 | GSTO1_HUMAN | Glutathione | GSTO1 | 4 | 25 | 5.81 | 2.482 | 2.89E−10 | 2.77E−06 | 0.395 |
| | | S-transferase | | | | | | | | |
| | | omega-1 | | | | | | | | |
| P13667 | PDIA4_HUMAN | Protein | PDIA4 | 3 | 4 | 1.09 | 2.633 | 2.99E−02 | 6.60E−07 | 0.420 |
| | | disulfide-isomerase | | | | | | | | |
| | | A4 | | | | | | | | |
| P62277 | RS13_HUMAN | 40S ribosomal | RPS13 | 1 | 1 | 7.95 | 2.638 | 2.88E−02 | 6.27E−07 | 0.421 |
| | | protein S13 | | | | | | | | |
| Q13509 | TBB3_HUMAN | Tubulin beta-3 | TUBB3 | 4 | 19 | 4.00 | 2.664 | 4.21E−04 | 4.87E−07 | 0.426 |
| | | chain | | | | | | | | |
| P30048 | PRDX3_HUMAN | Thioredoxin-dependent | PRDX3 | 1 | 1 | 5.47 | 2.769 | 3.86E−02 | 1.80E−07 | 0.442 |
| | | peroxide reductase, | | | | | | | | |
| | | mitochondrial | | | | | | | | |
| Q01813- | PFKAP_HUMAN | ATP-dependent 6- | PFKP | 2 | 3 | 2.68 | 3.116 | 3.23E−02 | 6.75E−09 | 0.494 |
| [2] | | phosphofructokinase, | | | | | | | | |
| | | platelet type | | | | | | | | |
| P50213 | IDH3A_HUMAN | Isocitrate | IDH3A | 3 | 3 | 2.19 | 3.239 | 6.67E−03 | 2.14E−09 | 0.510 |
| | | dehydrogenase | | | | | | | | |
| | | [NAD] | | | | | | | | |
| | | subunit alpha, | | | | | | | | |
| | | mitochondrial | | | | | | | | |
| Q9Y6N5 | SQRD_HUMAN | Sulfide:quinone | SQRDL | 1 | 2 | 2.22 | 3.487 | 8.97E−03 | 2.17E−10 | 0.543 |
| | | oxidoreductase, | | | | | | | | |
| | | mitochondrial | | | | | | | | |
| P38606- | VATA_HUMAN | V-type proton | ATP6V1A | 2 | 2 | 2.43 | 4.833 | 1.38E−02 | 1.75E−15 | 0.684 |
| [2] | | ATPase catalytic | | | | | | | | |
| | | subunit A | | | | | | | | |

### 2.2 Gene Ontology Analysis

The 58 proteins previously identified were analyzed using PANTHER (Mi et al., 2013, Nucleic Acids Res. 41, D377-D386) and classifiied into the following gene ontology and PANTHER categories: Protein Family; Protein class; Molecular function; Biological process; Cellular Component and Pathway (data not shown). The main represented biological process categories are metabolism (30%), cellular process (21%), cellular component organization and biological regulation (10%), localization and developmental process (8%), response to stimuli (4%), multicellular organismal process and immune system process (3%) and biological adhesion (1%). Concerning Protein Class, dysregulated proteins belongs to the main following protein classes: Nucleic acid binding (25%), Cytoskeletal Protein (13%), enzyme modulator (12%), Oxidoreductase and signaling molecules (8%), Chaperone (6%), transferase and transcription factor (4%), extracellular matrix protein, hydrolase, carrier protein, membrane traffic protein, cell junction protein, kinase, isomerase and receptor (2%) (Graphical representation not shown).

### 2.3 Western Blot Analysis of Candidate Proteins Validates the Proteomic Analysis

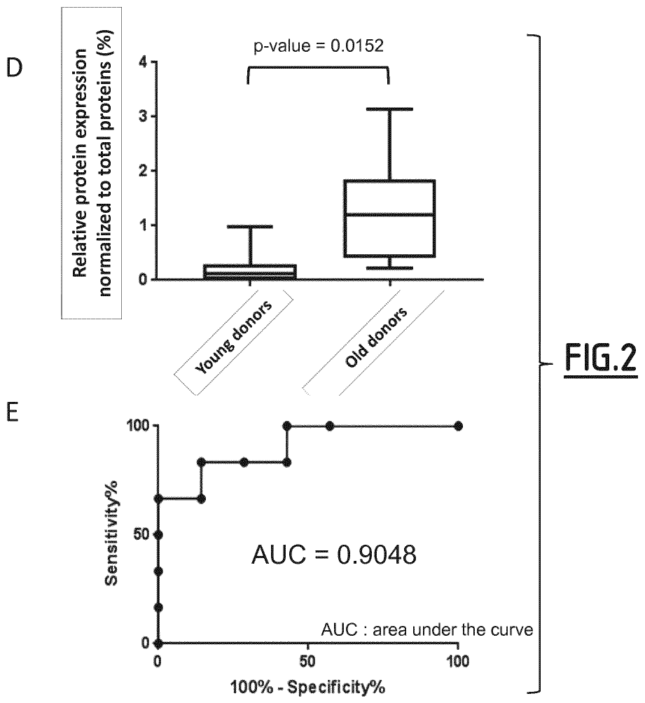

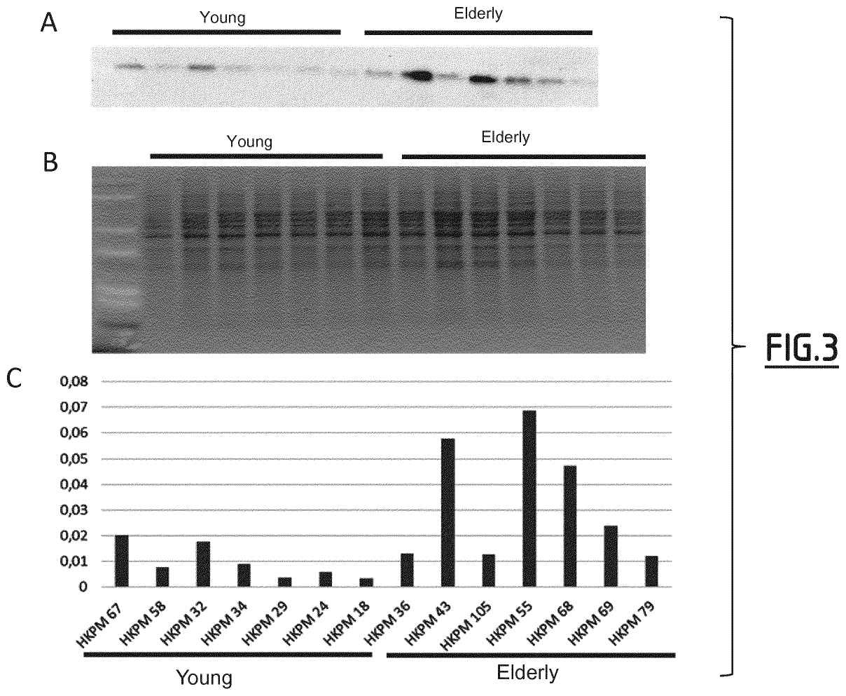

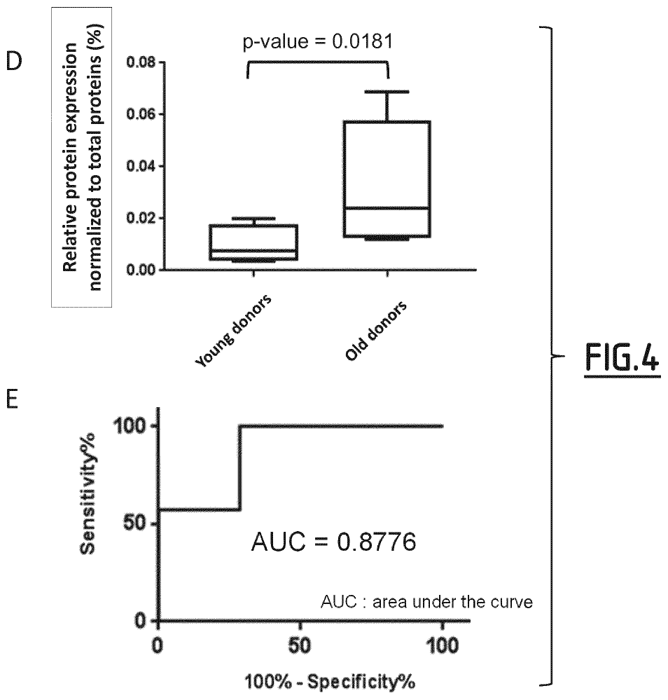

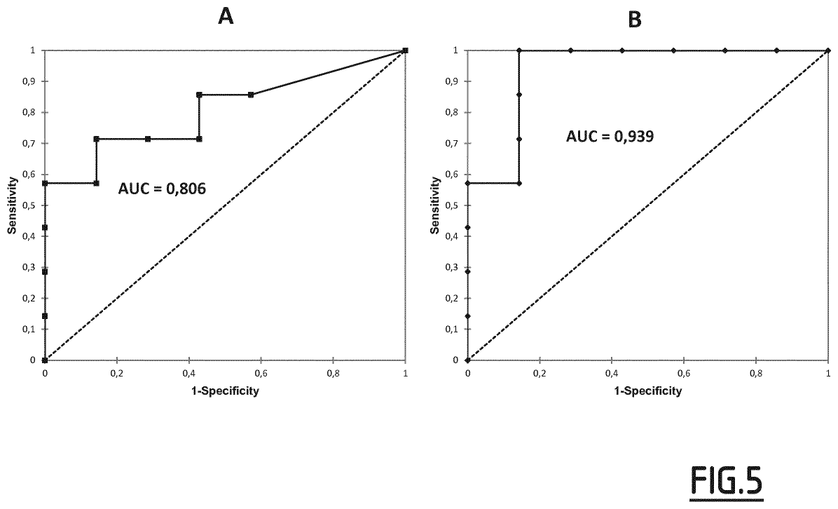

Two candidate proteins of interest were further analyzed by western blot on human primary keratinocytes cells from the same donors but also from ten other donors in order to validate the proteomic results on more donors to exclude the inter-individual variability. The selected proteins with the corresponding ratio obtained by proteomic experiment: Tubulin beta-3 chain (TBB3_HUMAN) ratio 2.6 and High mobility group protein HMGI-C (HMGA2_HUMAN) ratio 0.52. Western blots images and result of quantification are shown in FIGS. 2 and 4, respectively.

### 3. Discussion

Skin aging is a complex process with multifactorial origins that can be decipher using new technological approach such as quantitative proteomics. An iTRAQ-MALDI-TOF/TOF MS and MS/MS analysis was carried out to identify and quantify changes in human primary keratinocytes proteomes from young and elderly donors. 517 proteins were identified including proteins found mainly in keratinocytes such as Cornifin-B and Keratin-2e which is associated with keratinocyte activation, proliferation and keratinization (Collin et al., 1992, Exp. Cell Res. 202, 132-141). After applying robust statistical analysis, 58 proteins were found significantly differentially expressed depending on age status with 40 that were downregulated and 18 upregulated with aging.

The inventors found that more proteins are downregulated (40) than upregulated (18) with aging which is consistent with previous results from a gene expression study in women (Makrantonaki et al., 2012, PLoS ONE 7). The majority of proteins which expression is affected by age are involved in metabolism (30%) and nucleic acid binding (25%). Similar results have been observed in a previous transcriptomic study (Lener et al., 2006, Exp. Gerontol. 41, 387-397).

In this work, Cornifin-B has been found downregulated with aging as previously reported in two transcriptomic studies using women epidermis (Raddatz et al., 2013, Epigenetics Chromatin 6, 36) and skin biopsies (McGrath et al., 2012, Br. J. Dermatol. 166 Suppl 2, 9-15). Cornifin-B is a marker of keratinocyte differentiation (Tesfaigzi and Carlson, 1999, Cell Biochem. Biophys. 30, 243-265) and a downregulation of keratinocytes differentiation was already observed with aging (Raddatz et al., 2013, Epigenetics Chromatin 6, 36).

Peroxiredoxin 3 (PrxIII), a mitochondrial member of the antioxidant family of thioredoxin (Trx) peroxidases was found upregulated with aging in our study. Two other family members, Peroxiredoxin 1 and 2 were also upregulated in a previous report (Laimer et al., 2010, Exp. Dermatol. 19, 912-918). Peroxiredoxins are important cellular antioxidant, indeed they act as hydrogen peroxide and organic hydroperoxide scavengers (Nystrom et al., 2012, Genes Dev. 26, 2001-2008). It is well establish that with age, there is an increase in reactive oxygen species (ROS) production and a decrease in antioxidant activity both contributing to chronological aging (Poljšak et al., 2012, Acta Dermatovenerol. Alp. Pannonica Adriat. 21, 33-36). In oxidative stress conditions, PrxIII undergo overoxidation and subsequent irreversible inactivation. And it has been shown that in rats, this modified PrxIII form accumulate with aging (Musicco et al., 2009). In the present analysis, the peptide containing the cysteine that is overoxidized to sulfonic acid was not identified and therefore the two forms were not discriminated, explaining why in consequence a global upregulation of said protein was observed.

6-phosphofructokinase, platelet type (PFKAP_HUMAN) is upregulated with aging in our study. This enzyme catalyzes the phosphorylation of D-fructose 6-phosphate to fructose 1,6-bisphosphate by ATP, the first committing step of glycolysis. It has been shown that human primary keratinocytes derived from elderly donors show higher glucose uptake and increased lactate production, which are the indicators of a shift in metabolism towards increased glycolysis (Prahl et al., 2008, BioFactors Oxf. Engl. 32, 245-255). Thus the observed upregulation of PFKAP is correlated with the increased glycolysis in primary keratinocytes.

Comparing our results with other studies aiming at identify biomarker of skin aging show some differences that may be explained by different type/origin of skin samples, gender, difference in sample processing all along the workflow and the variable correlation between mRNA and protein expression levels (Schwanhäusser et al., 2011, Nature 473, 337-342).

Defining the differential protein signature with aging even if these changes could be initiating, adaptive or compensatory events is crucial to further our knowledge of skin aging. This study brings a new effort to reach a better understanding of the biology of skin aging and to identify new and specific targets that could help to diagnose, prevent and treat skin aging and associated pathologies.

### Example 2

Using the methods disclosed in Example 1 above, the inventors further demonstrated the improved sensitivity and specificity obtained when using a combination of markers of the invention.

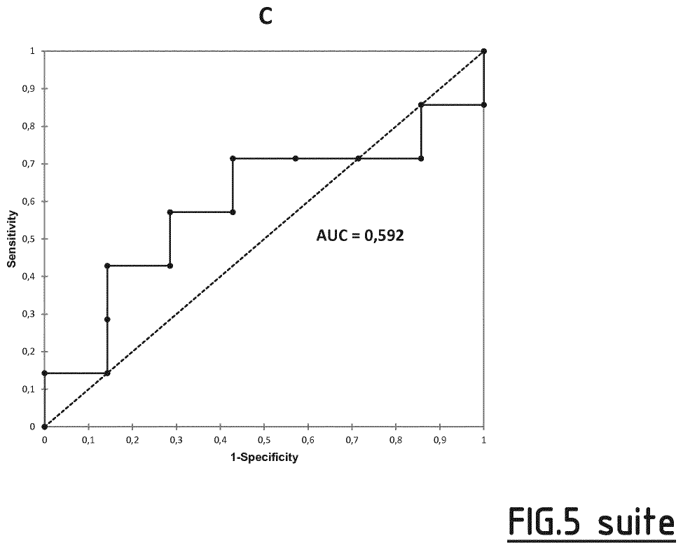

FIGS. 5A, B and C respectively show an area under the curve, respectively for beta-tubulin, HMGA2 and HMGN1, of 0.806, 0.939 and 0.592.

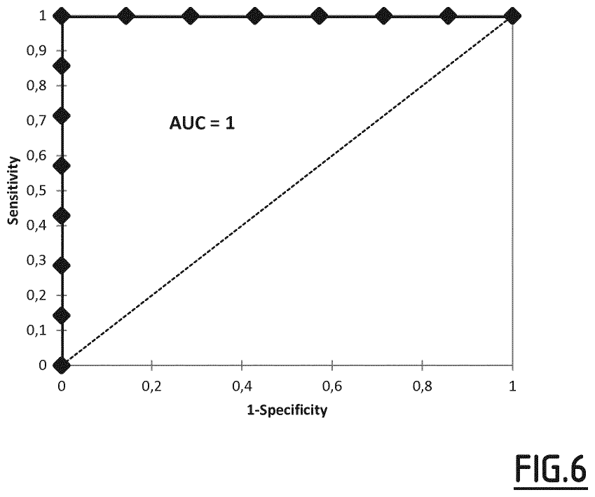

However, as shown in FIG. 6, when beta-tubulin, HMGA2 and HMGN1 are used in combination, it is possible to achieve a sensitivity and specificity of 100%, corresponding to an area under the curve of 1. The model equation used for obtaining such a sensitivity and specificity was as follows:

Prediction=83.76−(992.91×TUBULIN)−(4936.61×HMGA2)+(273.40×HMGN1)

1. An in vitro method to determine if the skin of a subject presents signs of physiological skin aging comprising the steps of
a) determining in a skin sample of said subject the expression level of a first protein encoded by a gene selected from the group of genes consisting of TUBB3, HMGA2 and HMGN1 and the expression level of at least one further protein encoded by a gene selected from the group of genes consisting of HMGN1, HIST1H2BK, PPFIA2, COXSA, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7, TUBB3, HMGA2, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13,
b) determining if the skin presents signs of physiological skin aging.

2. Method according to claim 1, further comprising a step of comparing the expression level of said first protein with a reference level and comparing the expression level of the at least one further protein with a reference level.

3. A method of cosmetic treatment capable of reducing or reversing the visible signs of physiological skin aging on a subject comprising the steps of
a) determining in a skin sample of said subject the expression level of a first protein encoded by a gene selected from the group of genes consisting of TUBB3, HMGA2 and HMGN1 and the expression level of at least one further protein encoded by a gene selected from the group of genes consisting of HMGN1, HIST1H2BK, PPFIA2, COXSA, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7, TUBB3, HMGA2, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13,
b) deducing from the expression level of said first protein and the expression level of said at least one further protein determined in step a) if the skin presents signs of physiological skin aging, and
c) if the skin is determined as presenting signs of physiological aging, treating said subject with a cosmetic composition that reduces or reverses the visible signs of physiological skin aging.

4. A method to identify a substance that is capable of reducing or reversing the visible signs of physiological skin aging comprising the steps of
a) treating a skin sample with a candidate substance,
b) determining in the skin sample of step a) the expression level of a first protein encoded by a gene selected from the group of genes consisting of TUBB3, HMGA2 and HMGN1 and the expression level of at least one further protein encoded by a gene selected from the group of genes consisting of HMGN1, HIST1H2BK, PPFIA2, COXSA, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7, TUBB3, HMGA2, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13,
c) comparing the expression level of said first protein and said at least one further protein with the expression level of said first protein and said at least one further protein in a skin sample that has not been treated with said candidate substance,
d) identifying the candidate substance as a substance that reduces or reverses the visible signs of physiological skin aging.

5. Method according to claim 4, wherein the skin sample of step a) and the skin sample that has not been treated with said candidate substance of step c) are exposed to an environment that induces skin aging.

6. Method according to any one of claims 1 to 5, wherein said skin sample comprises keratinocytes, preferably primary keratinocytes.

7. Method according to any one of claims 1 to 6, wherein said subject is caucasian.

8. Method according to any one of claims 1 to 7 wherein said subject is a woman.

9. Method according to any one of claims 1 to 2 and 4 to 8, wherein the expression level of a first protein and at least one further protein is compared with a reference expression level by determining a ratio of expression of said first protein by dividing the expression level of said first protein through a reference expression level of said first protein and by determining a ratio of expression of said at least one further protein by dividing the expression level of said at least one further protein through a reference expression level of said at least one further protein.

10. Method according to claim 9, wherein the skin of said subject presents signs of physiological aging, when the ratio of expression is higher or lower than 1 for the first protein encoded by a gene selected from the group of genes consisting of TUBB3, HMGA2 and HMGN1.

11. Method according to claim 9 or 10, wherein the skin of said subject presents signs of physiological aging, when the ratio of expression is higher than 1 for the at least one further protein encoded by a gene selected from the group of genes consisting of TUBB3, HMGA2, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13.

12. Method according to claim 9 or 10, wherein the skin of said subject presents signs of physiological aging, when the ratio of expression is less than 1 for the at least one further protein encoded by a gene selected from the group of genes consisting of HMGN1, HIST1H2BK, PPFIA2, COXSA, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7.

13. A kit comprising
at least one capture ligand for determining the expression level of a first protein encoded by a gene selected from the group of genes consisting of TUBB3, HMGA2 and HMGN1, and
at least one capture ligand for determining the expression level of at least one further protein encoded by a gene selected from the group of genes consisting of HMGN1, HIST1H2BK, PPFIA2, COXSA, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7, TUBB3, HMGA2, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13.

14. A kit according to claim 13, wherein the capture ligand is an antibody.

15. Use of a kit as defined in claims 13 and 14 for determining in a skin sample the expression level of one first protein encoded by a gene selected from the group of genes consisting of TUBB3, HMGA2 and HMGN1, and
the expression level of at least one further protein encoded by a gene selected from the group of genes consisting of HMGN1, HIST1H2BK, PPFIA2, COXSA, MT1E, HMGN2, EEA1, CDV3, ZC3H11A, HMGA1, PTMA, SERBP1, PDAP1, TMSB10, PSMD9, CALM1, MT1G, TPM4, SPRR1B, GBF1, HDGF, HSPE1, DBI, TRIM6, PTMS, IMUP, SH3BGRL3, RPS28, STMN1, AHSG, PFDN6, SUMO2, PFDN2, NEB, HMGB1, TMSB4X, CLTB, MYH11, SRSF7, TUBB3, HMGA2, ATP6V1A, SQRDL, IDH3A, PFKP, PRDX3, RPS13, PDIA4, GSTO1, GSTP1, ACTR3, SLC2A1, CCT5, PSMB2, PLS3, PSMD2, IGHG4, RPL13.
